(19) United States
(12) Patent Application Publication
(10) Pub. No.: US 2007/0020652 A1
(43) Pub. Date: Jan. 25, 2007

(54) Methods and compositions for RNA interference

(75) Inventors: A. Sanchez Alvarado, Salt Lake City, UT (US); Peter W. Reddien, Cambridge, MA (US); Adam L. Bermange, London (GB)

Correspondence Address: TRASK BRITT, P.O. BOX 2550, SALT LAKE CITY, UT, 84110, US

(73) Assignee: University of Utah Research Foundation, Salt Lake City, UT (US)

(21) Appl. No.: 11/413,795

(22) Filed: Apr. 28, 2006

Related U.S. Application Data
(63) Continuation of application No. PCT/US04/37475, filed on Nov. 10, 2004.
(60) Provisional application No. 60/518,856, filed on Nov. 10, 2003.

(51) Int. Cl. C40B 30/06; C40B 40/08; C12N 15/09; C07H 21/02

(52) U.S. Cl. 435/6; 435/455; 435/325; 536/23.1

(57) ABSTRACT

The invention relates to improved methods of attenuating expression of a target gene in a eukaryotic cell with dsRNA, identifying nucleic acid sequences responsible for conferring a particular phenotype to a cell, alleviating pest infestation in plants, and altering gene expression in an undifferentiated stem cell or the differentiated progeny thereof. Transcription of the RNA, which will form the dsRNA, is terminated by one or more terminators sequences, thereby increasing the efficiency of inhibition.

### CROSS-REFERENCE TO RELATED APPLICATIONS

This application is a continuation of PCT International Patent Application No. PCT/US04/037475, filed on Nov. 10, 2004, designating the United States of America, and published, in English, as PCT International Publication No. WO 2005/047300 A2 on May. 26, 2005, and also claims the benefit under 35 U.S.C. §119(e) to U.S. Provisional Patent Application Ser. No. 60/518,856, filed Nov. 10, 2003, the entire contents of each of which are hereby incorporated herein by this reference.

### TECHNICAL FIELD

The invention relates to biotechnology generally, and, more particularly, to ways of improving the efficiency of double stranded RNA (“dsRNA”) inhibition as a method of inhibiting gene expression in eukaryotes. In particular, the invention relates to the addition of terminator sequences to the vectors used to express dsRNA to enhance inhibition of gene expression by dsRNA.

### BACKGROUND

The mid-1980s presented a potential new avenue for therapeutic intervention with the discovery of antisense technologies wherein target mRNA transcripts hybridize in a sequence-specific manner to homologous RNA, DNA or chemically altered nucleic acids, thereby inhibiting their expression (Dean, N. M., Functional genomics and target validation approaches using antisense oligonucleotide technology, 12 Curr. Opin. Biotechnol 622 (2001)) post-transcriptionally. In theory, this type of approach could selectively silence any gene product before it was translated, and was therefore received with great enthusiasm. Unlike classical small molecules, however, antisense nucleic acids have molecular weights greater than 1000 Daltons (Da) which have resulted in significant delivery problems.

In the early 1990s, nucleic acid molecules were used to directly target the transcriptional regulation of gene expression. “Triplex” generating reagents opened the window for researchers to inhibit the transcription process itself by introducing a nucleic acid molecule that hybridizes to a specific sequence of DNA within a cell to block cellular machinery from acting to initiate or elongate gene transcription (Casey, B. P. and P. M. Glazer, Gene targeting via triple-helix formation, 67 Prog. Nucleic Acid Res. Mol. Biol. 192 (2001)). Like antisense, however, delivery issues and transitory inhibitory effects have limited the success of this strategy.

Double-stranded RNA methods of inhibiting gene expression, called, e.g., RNA interference (“RNAi”), RNA silencing, post-transcriptional gene silencing, and quelling (U.S. Pat. Appl. Pub. No. 2003/0084471 A1, the contents of which are incorporated by this reference), are considered substantially more effective than merely providing an RNA strand individually as proposed in antisense technology. RNAi is an innate cellular process activated when a dsRNA molecule of greater than 19 duplex nucleotides enters the cell, causing the degradation of not only the invading dsRNA molecule, but also single-stranded RNAs of identical sequences, including endogenous mRNAs.

RNAi methods are based on nucleic acid technology. However, unlike antisense and triplex approaches, the dsRNA activates a normal cellular process leading to a highly specific RNA degradation, and a cell-to-cell spreading of this gene silencing effect. Injection of dsRNA, for example, acts systemically to cause post-transcriptional depletion of the homologous endogenous RNA in Caenorhabditis elegans (Fire et al., Potent and specific genetic interference by double-stranded RNA in Caenorhabditis elegans, 391 Nature 806 (1998); Montgomery et al., RNA as a target of double-stranded RNA-mediated genetic interference in Caenorhabditis elegans, 95 Proc. Natl. Acad. Sci. 15502 (1998)). This depletion of endogenous RNA causes effects similar to a conditional gene “knock out,” revealing the phenotype caused by the lack of a particular gene function.

Planarians are bilaterally symmetric metazoans renowned for their regenerative capacities, extensive tissue turnover and regulation as part of their normal homeostasis, and the presence of a pluripotent adult stem cell population known as the neoblasts. These prominent attributes of normal planarian biology relate to classic problems of developmental biology and in vivo stem cell regulation that cannot be readily investigated in other commonly studied organisms^{1,2}.

Given that these problems are poorly understood and are of importance to the life of most metazoans, a strategy was devised to uncover their genetic regulation in the planarian Schmidtea mediterranea. How can the function of genes regulating planarian biology be explored? One approach that has been pivotal in understanding the biology of multiple metazoans, including Drosophila melanogaster³, Caenorhabditis elegans⁴, and Danio rerio^{5,6}, involves large scale functional genetic surveys. Such an approach has been precluded by planarian life cycles. The development of dsRNA-mediated genetic interference (RNAi)⁷ and the application of RNAi to systematic studies of gene function^{8,10} has opened the door for a new generation of genetic manipulations. Here, 1065 genes were selected with a intention of representing sampling of the planarian S. mediterranea genome, and developed a large-scale, RNAi-based screening strategy to systematically disrupt their expression and assess their function in planarian biology. This screen is the first of its kind and defines the major phenotypic categories that exist in planarians following gene perturbation.

However, RNAi using dsRNA generated by vectors currently known in the art may only weakly elicit phenotypic expression, or may result in only some of the subject organisms expressing the expected phenotype.

### SUMMARY OF THE INVENTION

The invention relates to an improved method of attenuating expression of a target gene in a eukaryotic cell. This method involves introducing dsRNA into the cell in an amount sufficient to attenuate expression of the target gene, wherein the dsRNA includes a nucleotide sequence that hybridizes under stringent conditions to a nucleotide sequence of the target gene, and where the dsRNA is expressed from a vector containing one or more transcriptional regulators, which includes one or more transcription terminator.

The invention also relates to a method of attenuating expression of a target gene in a eukaryotic cell. This method involves introducing into the cell an expression vector having at least one nucleotide sequence similar to the target gene, which, when transcribed, produces dsRNA in an amount sufficient to attenuate expression of the target gene.

Another aspect of the invention relates to a method of attenuating expression of a target gene in a eukaryotic cell. The method involves introducing into the cell an expression vector having two promoters positioned on opposite strands of the nucleic acid duplex, such that, upon binding of an appropriate transcription factor to the promoters, the promoters are capable of initiating transcription of a target nucleotide sequence that is cloned between the promoters, to generate dsRNA in an amount sufficient to attenuate expression of the target gene.

Yet another aspect of the invention relates to a method of attenuating expression of a target gene in a cell. This method involves introducing into the cell a hairpin nucleic acid in an amount sufficient to attenuate expression of the target gene, where the hairpin nucleic acid includes an inverted repeat of a nucleotide sequence that hybridizes under stringent conditions to a nucleotide sequence of the target gene. In this and all aspects of the present invention involving a hairpin nucleic acid, the hairpin nucleic acid may be, without limitation, RNA.

The invention also relates to a hairpin nucleic acid for inhibiting expression of a target gene. This hairpin nucleic acid has a first nucleotide sequence that hybridizes under stringent conditions to a nucleotide sequence of the target gene; and a second nucleotide sequence which is a complementary inverted repeat of the first nucleotide sequence and which hybridizes to the first nucleotide sequence to form a hairpin structure.

Still another aspect of the present invention relates to a method of identifying nucleic acid sequences responsible for conferring a particular phenotype in a cell. This method involves constructing a library of nucleic acid sequences from a cell in an orientation relative to at least one promoter to produce dsRNA; introducing the dsRNA library into a target cell; identifying members of the library which confer a particular phenotype on the cell; and identifying the nucleotide sequence of the cell which corresponds to the library member conferring the particular phenotype.

Yet another aspect of the invention relates to a method of conducting a drug discovery business. This method involves identifying by the subject assay a target gene that provides a phenotypically desirable response when inhibited by RNAi; identifying agents by their ability to inhibit expression of the target gene or the activity of an expression product of the target gene; conducting therapeutic profiling of agents identified in the immediately prior step, or further analogs thereof, for efficacy and toxicity in cells; and formulating a pharmaceutical preparation including one or more agents identified in the immediately prior step as having an acceptable therapeutic profile.

Another aspect of the invention relates to a method of conducting a target gene discovery business. This method involves identifying by the subject assay a target gene that provides a phenotypically desirable response when inhibited by RNAi; optionally conducting therapeutic profiling of the target gene for efficacy and toxicity in cells; optionally licensing, to a third party, the rights for further drug development of inhibitors of the target gene; and developing a drug to inhibit expression of the target gene.

The invention also relates to transgenic eukaryotes, which include a transgene encoding a dsRNA construct.

Another aspect of the invention relates to a dsRNA for inhibiting expression of a eukaryotic gene. This dsRNA includes a first nucleotide sequence that hybridizes under stringent conditions to a second nucleotide sequence, which is complementary to the first nucleotide sequence.

Yet another aspect of the invention relates to a method of alleviating pest infestation of plants. This method involves identifying a DNA sequence of the pest that is critical for the pest's survival, growth, proliferation or reproduction; cloning the sequence or a fragment thereof into a vector capable of transcribing the sequence and its complement to produce dsRNA; and introducing the vector into the plant under conditions effective to alleviate the pest infestation.

The invention further relates to a therapeutic method for alleviating parasitic infestation (e.g., helminth) of animals or humans. This method involves identifying a DNA sequence of the parasitic pest that is critical for the pest's survival, growth, proliferation or reproduction; cloning the sequence or a fragment thereof into a vector capable of transcribing the sequence and its complement to produce dsRNA; and introducing the vector into the animal or human under conditions effective to alleviate the pest infestation. For example, a method of alleviating parasitic helminthic infections in humans and animals is provided. In this example, a DNA sequence critical for the pest's survival, growth, proliferation or reproduction which is preferably not found in the genome of humans or animals to be treated may be cloned into a vector capable of transcribing the sequence and its complement to produce dsRNA and introduced into the infected hosts under conditions effective to alleviate the pest infestation.

The invention yet further relates to a method of treating a subject, either plant or animal, infected by parasitic pets (e.g., helminthes). Wherein infection of a subject by helminthes is reduced according to the invention.

The invention also relates to the plasmid identified as pDONR dT7. In another aspect, the invention relates to a library of RNAi entry clones originating from a eukaryotic cell, such as a planarian, and further to methods of screening with the library.

Another aspect of the invention relates to an expression vector. This vector includes one or more promoters oriented relative to a polynucleotide sequence, for example a DNA molecule, such that the promoters are capable of initiating transcription of the polynucleotide sequence of interest, wherein at least one transcription terminator sequence is located 3′ of the polynucleotide sequence of interest, to produce dsRNA. When the complement of a termination sequence also functions as a terminator sequence, it is necessary to place the termination sequence 5′ of the promoter, as defined on the complementary strand.

The invention also relates to a method of altering gene expression in an undifferentiated stem cell or the differentiated progeny thereof. This method involves introducing into the cell one or more dsRNAs according to the invention under conditions effective to alter gene expression in the cell.

In yet an additional embodiment, the invention relates to a method of identifying a function in a gene in a planarian. The method involves producing a library of genes in a bacterial cell population, feeding the bacterial cell population to the planarian, and observing a change in a phenotype or behavior (e.g., changes at a cellular level). The identity of the gene producing the change in the phenotype or the change at the cellular level may be determined or sequenced.

Thus, in another embodiment, the instant invention is directed towards nucleic acids or sequences identified with the method of identifying the function of the gene of the instant invention.

In another aspect, the invention relates to a method of screening for compounds that are involved in the pathogenesis of a cell. The method includes subjecting the cell to a stress, such as an infection, and altering gene expression in the cell using RNAi. The cell is observed for changes in phenotype or a change at the cellular level in response to the stress.

The invention may be used, inter alia, in all applications that employ RNAi, including, but not limited to, genomic analysis and gene-silencing therapies.

The invention may be used, for example, to provide efficient dsRNA production; improve the strength of phenotypic expression and the number of individuals expressing a target phenotype; and streamline the production of dsRNA-producing plasmids for a large number of genes. The invention is useful, inter alia, as a research tool and for disease therapies including, reduction or inhibition of aberrant transcripts and translation products resulting from chromosomal translocations, deletions, and other mutations, and inhibition of viral products such as the HIV genome or specific products such as RCV. The invention is useful in all organisms in which RNAi is effective. The invention is also useful in all applications that employ RNAi. In addition, the invention is useful in all business practices utilizing RNAi.

### BRIEF DESCRIPTION OF THE DRAWINGS

FIG. 1 is a schematic diagram depicting an overview of the RNAi pathway.

FIG. 2 is a schematic diagram showing the RNAi vectors L4440 and pDONRdT7.

FIGS. 3A-3D. RNAi screening strategy in S. mediterranea. FIG. 3A. S. mediterranea cDNAs were transferred into pDONRdT7, containing two T7 promoters and terminators, using a single-step Gateway (Invitrogen) reaction (see methods). FIG. 3B. RNAi screening procedure involved expressing dsRNA in bacteria, mixing bacteria with an artificial food mixture, feeding the planarians a total of three times, amputating the planarians twice, two rounds of regeneration, and three scorings (see Example 5). FIG. 3C. Animals with a phenotype were labeled with αH3P (mitotic neoblasts) and VC-1 (photoreceptor neurons). Animals with no phenotype were labeled with VC-1 and screened for phenotypes. FIG. 3D. 143 genes that conferred phenotypes following RNAi and amputation were inhibited by RNAi. Tissue homeostasis was observed in a process involving five feedings and scoring for six weeks. Presence and capacity to divide of neoblasts was assessed by amputation, fixation, and labeling with αH3P (see Examples 5 and 6).

FIGS. 4A-4J. Representative phenotypes from the RNAi screen. Phenotype nomenclature and homologies for representative genes can be found in Table 4 of the incorporated PCT International Application PCT/US2004/037475 (corresponding to PCT International Publication WO 2005/047300 A2 of May. 26, 2005). White arrowheads indicate defects. Anterior, left. v, ventral surface. Bar, 0.2 mm. FIG. 4A. Control, unc-22 RNAi animal. Irradiation at 6000 rad blocked regeneration (BLST(0), 8d) and caused curling (CRL,15d). Black arrowhead, photoreceptor. P, pharynx. Brackets, blastema (unpigmented). FIG. 4B. Reduced regeneration, curling, and caudal regeneration defects. FIG. 4C. Pointed, wide, and indented blastemas. FIG. 4D. Diffuse, faint, and asymmetric photoreceptors. FIG. 4E. Regression of the anterior tip and between the photoreceptors. FIG. 4F. Lesions and lysis. FIG. 4G. Bloated and blistered. FIG. 4H. Sticking and stretching and hourglass postures. FIG. 4I. Spots and pigment freckles. FIG. 4J. Growth and bump.

FIGS. 5A-5N. Cellular analyses of regeneration abnormalities. Anterior, left. FIGS. 5B-5L. Representative defects observed with VC-1 staining. Arrowheads, abnormalities. Bar, 0.1 mm. oc, optic chiasmata. cb, cell bodies. The cephalic ganglia of H.68.4a RNAi animals were also labeled with α-synaptotagmin. The nomenclature system is similar to that used in Table 3 and FIG. 5. Phenotype terms: EXTNT, photoreceptor regeneration extent abnormal. EXTNT descriptors: nopr, no labeling; ltd, limited; sqish, slightly underdeveloped. PRCELLS, photoreceptor cell bodies abnormal. Descriptors: wd, photoreceptors wide; difus, diffuse clustering; asym, asymmetry; trs, tears, ectopic neurons posterior to cluster; ecto, ectopic photoreceptor. DISORG, axon disorganization. No descriptor applied if general and/or variable. Descriptors: straightoc, oc straight; splitoc, axons fail to cross midline; fwdproj, cell body projections toward anterior tip; ectoax, extra projections. FIG. 5M. αH3Plabeling summary of animals from RNAi of 140 genes. 14d, 14 days. Bar, 1 mm. Irradiated animals received 6000 rads. Control unc-22 RNAi animals had an average of 212±37 cells/mm length (from photoreceptors to tail). Defects were categorized as LOW(v), LOW, LOW(s), normal, HIGH(s), HIGH, and HIGH(v) (“v,” very; “s,” slightly). The LOW(s) threshold is set at the control mean less 2× the standard deviation (sd). This absolute value was divided into three equal ranges to set LOW and LOW(v). The same ranges added to the mean plus 2× sd set the high ranges. For those within 2× sd but visually abnormal, data were considered significant if P<0.01(t-test). FIG. 5N. αH3P-labeling summary of RNAi animals fixed 16 or 24 h after amputation (see text for gene details). Animals were fixed after the first or second amputation as appropriate (FIG. 3). Control animals were fed or starved, as appropriate, and fixed at 16 h or 24 h. Bar, 1 mm. Data were categorized as described in FIG. 5M. A complete table of results can be found in Table 8 of the incorporated PCT International Application PCT/US2004/037475 (corresponding to PCT International Publication WO 2005/047300 A2 of May. 26, 2005).

FIGS. 6A-G. Representative defects in intact animals following RNAi. FIGS. 6A-G. Anterior, left. Arrowheads, defects. v, ventral. Bar, 0.4 mm. Nomenclature similar to Table 4 of the incorporated PCT International Application PCT/US2004/037475 (corresponding to PCT International Publication WO 2005/047300 A2 of May. 26, 2005). Additional phenotype terms: CONSTR, constriction. Additional descriptors and modifiers: (i) Body regions: all, entire animal; hd, head; hdside, head lateral edge; ant, anterior half or the anterior end of a region; bm, brain, posterior to photoreceptors; tl, tail; int, gastrovascular system. (ii) Lesions; big, large; many, multiple; bli, blistered; strp, strip. FIG. 6A. unc-22 RNAi animals, negative control. Irradiation at 6000 rads caused tissue regression (8d) and curling (15d). FIG. 6B. Regression. FIG. 6C. Curling. FIG. 6D. Tumorous and blistered lesions. FIG. 6E. Lesions and lysis. FIG. 6F. Lesions in the pattern of photoreceptor neurons and gastrovascular system. FIG. 6G. Lesions at specific body locations.

FIGS. 7A-E. Distribution of RNAi phenotypes. FIGS. 7A-D. Each square represents observations from the RNAi of a single gene. The square location for a given gene is the same in each panel. Y axis, blastema size with 3=normal and 0=no regeneration. X axis, number of cells labeled with αH3P (described in FIG. 3). FIG. 7A. Colors represent distribution of curling following amputation. FIG. 7B. Colors represent defects seen in intact RNAi animals. FIG. 7C. Colors represent regression and curling defects seen in intact RNAi animals. FIG. 7D. Colors represent lesion formation in intact RNAi animals. FIG. 7E. Groups of genes that share profiles of defects are summarized. Some genes are found in multiple categories. LYS, lysis. Reg, regeneration (blastema formation); “abort” indicates too small or no blastema formed. CRL, curling. BLST, blastema. VC-1, abnormal photoreceptor neurons (see text, Table 7 of the incorporated PCT International Application PCT/US2004/037475 (corresponding to PCT International Publication WO 2005/047300 A2 of May. 26, 2005)). PHX, pharynx regeneration in tail fragments. RGRS, tissue regression. BHV, behavior abnormal. H3P categorization is described in FIG. 3 and Table S3. For the profile in which animals CRL and/or RGRS and have normal numbers of αH3P-labelled cells, those genes associated with low mitotic numbers 14d after regeneration were excluded. LES; lesions. Genes are categorized as novel if they have no predicted function. Genes are characterized as specific if they are predicted to encode proteins involved in signal transduction, transcription, cell adhesion, neuronal functions, disease, RNA binding, channels/transporter function, cytoskeletal regulation. Genes are characterized as basal if they are predicted to encode proteins involved in translation, metabolism, RNA splicing, proteolysis, protein folding, vesicle trafficking, cell cycle, or cytoskeleton machinery.

### DETAILED DESCRIPTION OF THE INVENTION

The invention relates to a method of attenuating expression of a target gene in a eukaryotic cell. Double-stranded RNAs (dsRNAs) can provoke gene silencing in numerous in vivo contexts including Drosophila, Caenorhabditis elegans, planaria, hydra, trypanosomes, fungi, plants and other eukaryotic cells. The method includes introducing double stranded RNA into the cell in an amount sufficient to attenuate expression of the target gene, where the dsRNA includes a nucleotide sequence that hybridizes under stringent conditions to a nucleotide sequence of the target gene, and where the dsRNA is expressed from a vector containing one or more transcription terminators.

As used herein, “target gene” includes any nucleotide sequence, which may or may not contain identified gene(s), including, without limitation, intergenic region(s), non-coding region(s), untranscribed region(s), intron(s), exon(s), and transgene(s).

dsRNA activates a normal cellular process leading to a highly specific RNA degradation, and a cell-to-cell spreading of this gene silencing effect in several RNAi models. (Shuey, et al, RNAi: gene-silencing in therapeutic intervention, 7(20) Drug Discovery Today 1040 (2002).) Injection of dsRNA, for example, acts systemically to cause post-transcriptional depletion of the homologous endogenous RNA in C. elegans (U.S. Pat. Appl. Pub. No. 2003/0084471 A1, the contents of which are incorporated by this reference). This depletion of endogenous RNA causes effects similar to a conditional gene ‘knock out,’ revealing the phenotype caused by the lack of a particular gene function. C. elegans nematodes can, for example, be fed with bacteria engineered to express dsRNA corresponding to a C. elegans target gene. Nematodes fed with engineered bacteria show a phenotype similar to mutants containing a mutation in the target gene (1998 Nature 395: 854).

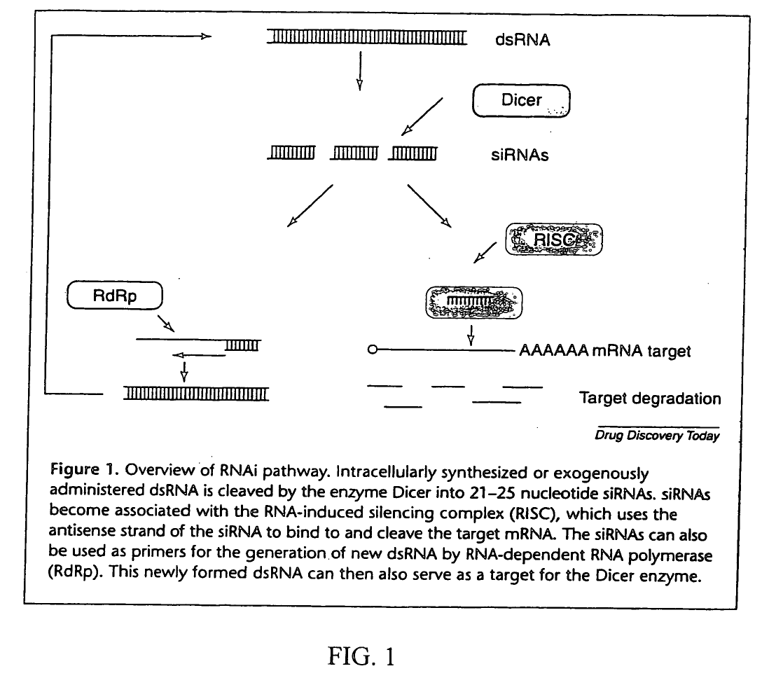

FIG. 1 is a schematic diagram depicting an overview of the RNAi pathway. Intracellular synthesized or exogenously administered dsRNA is cleaved by an enzyme, for example, Dicer, into siRNAs approximately 19 to about 25 nucleotides in length. siRNAs become associated with the RNA-induced silencing complex (RISC), which uses the antisense strand of the siRNA to bind to the target mRNA, with cleavage of the mRNA. The siRNAs can also be used as primers for the generation of new dsRNA by RNA-dependent RNA polymerase (RdRp). This newly formed dsRNA can then also serve as a target for the Dicer enzyme.

In both plant and animal cells, intracellular exposure of a dsRNA sequence can result in the specific post-transcriptional gene silencing (“PTGS”) of the homologous cellular RNA (Fire, A. et al. Potent and specific genetic interference by double-stranded RNA in Caenorhabditis elegans, 391 Nature 806 (1998); Shuey, et al., RNAi: gene-silencing in therapeutic intervention, 7(20) Drug Discovery Today 1040 (2002)). The RNAi pathway, as shown in FIG. 1, consists of the presentation of a “triggering” dsRNA that is subsequently processed into siRNAs by an RNaseIII-like enzyme, for example, Dicer (Zamore, P. D. et al., RNAi: double-stranded RNA directs the ATP-dependent cleavage of mRNA at 21 to 25 nucleotide intervals, 101 Cell 25(2000); Hutvagner, G. and Zamore, P. D., RNAi: nature abhors a double-strand, 12 Curr. Opin. Genet. Dev. 225 (2002)). This siRNA species, which may be about 19 to about 25 bp in length, is then incorporated into a multi-subunit RNA-induced silencing complex, which targets the unique cellular RNA transcript for enzymatic degradation. RNA hydrolysis occurs within the region of homology directed by the original siRNA (Fibashir, S.M. et al., RNA interference is mediated by 21 and 22 nucleotide RNAs, 15 Genes Dev. 188 (2001)), thereby selectively inhibiting target gene expression.

dsRNA also activates RNA-dependent RNA polymerase (RdRp)-mediated generation and amplification of single-stranded RNA into dsRNA precursors (Ahlquist, P., RNA-dependent RNA polymerases, viruses, and RNA silencing, 296 Science 1270 (2002)), as shown in FIG. 1, thereby prolonging dsRNA's inhibitory effect.

Local exposure to dsRNA, which may be produced from a viral or plasmid vector producing dsRNA, is often followed by a widespread gene silencing effect throughout most, if not all, tissues of the exposed organism. This systemic RNAi-mediated gene silencing has been observed in, e.g., plants (Napoli, C. et al., Introduction of a chalcone synthase gene into Petunia results in reversible co-suppression of homologous genes in trans, 2 Plant Cell 279 (1990)), nematodes (Fire, A. et al. Potent and specific genetic interference by double-stranded RNA in Caenorhabditis elegans, 391 Nature 806 (1998); Tabara, H. et al., RNAi in C. elegans: soaking in the genome sequence, 282 Science 430 (1998); Timmons, L. et al., Ingestion of bacterially expressed CISRNAS can produce specific and potent genetic interference in Caenorhabditis elegans, 263 Gene 103(2001); Winston, W. M. et al., Systemic RNAi in C. elegans requires the putative transmembrane protein SID-1, 295 Science 2456 (2002)), planarians (Sanchez Alvarado et al., dsRNA Specifically Disrupts Gene Expression During Planarian Regeneration, 96 Proc. Natl. Acad. Sci. USA 5049 (1999); Cebria F. et al., FGFR-related gene nuo-darake restricts brain tissues in the head region of planarians, 419 Nature 620 (2002)), and mice (Pachuk C. J. et al., dsRNA mediated post-transcriptional gene silencing and the interferon response in human cells and an adult mouse model, Keystone Symposia; RNA Interference, Cosuppression and Related Phenomena, February 21-26, Taos, N. Mex. Abstract no. 217 (2002)), and is thought to involve at least two components: a previously described local and cellular PTGS effect, and a separate, but related global gene-silencing mechanism often referred to as transcriptional gene silencing (“TGS”). (Shuey, et al., RNAi: gene-silencing in therapeutic intervention, 7(20) Drug Discovery Today 1040 (2002).)

It has been shown that long transfected dsRNAs are processed into shorter siRNAs when introduced into the cell (Zamore, P. D. et al., RNAi: double-stranded RNA directs the ATP-dependent cleavage of mRNA at 21 to 23 nucleotide intervals, 101 Cell 25 (2000)). Chemically synthesized siRNAs have been used for RNAi (Elbashir, S. M. et al., Duplexes of 21-nucleotide RNAs mediate RNA interference in cultured mammalian cells, 411 Nature 494 (2001)). Different siRNAs and siRNA-expressing plasmids have shown varying abilities to induce RNAi for an identical target mRNA (Holen, T. et al., Positional effects of short interfering RNAs targeting the human coagulation trigger Tissue Factor, 30 Nucleic Acids Res. 1757 (2002); Lee, N. S. et al., Expression of small interfering RNAs targeted against HIV-1 rev transcripts in human cells, 19 Nat. Biotechnol. 500 (2002)). Suitable short dsRNAs may be designed by one of ordinary skill in the art, based on knowledge of the suppressive activities of individual siRNAs. Longer (>50 bp) dsRNA molecules may also be used to provide multiple Dicer-derived siRNAs to the cell, thus, allowing the cell to employ the endogenous dsRNA silencing pathway to choose the most effective silencing siRNA(s). This allows for the simultaneous expression of a large number of siRNAs that are derived from a single precursor dsRNA, some of which should elicit a strong and sequence-specific RNAi response without inducing a generalized suppressive or apoptotic response. A longer dsRNA would also permit targeting of more than one message with a single construct and could potentially alleviate the development of resistance to potential RNAi therapies that may result from, for example, point mutations in the target.

A person of ordinary skill in the art will understand that in animals exhibiting a PKR response (Stark, G. E. et al., How cells respond to interferons, 67 Annu. Rev. Biochem. 227 (1998); Gil, J. and Esteban, M., Induction of apoptosis by the dsRNA dependent protein kinase (PKR): mechanism of action, 5 Apoptosis 107 (200)), the response, where desirable and appropriate, may be avoided or overcome. For example, the PKR pathway may be circumvented with the use of smaller dsRNAs. (Shuey, et al., RNAi: gene-silencing in therapeutic intervention, 7(20) Drug Discovery Today 1040 (2002).) Vector-mediated delivery of larger dsRNAs can also circumvent the PKR response (Shuey, et al., RNAi: gene-silencing in therapeutic intervention, 7(20) Drug Discovery Today 1040 (2002); Pachuk C. J., et al., dsRNA mediated post-transcriptional gene silencing and the interferon response in human cells and an adult mouse model, Keystone Symposia, RNA Interference, Cosuppression and Related Phenomena, February 21-26, Taos, N. M. Abstract No. 217 (2002)). Additionally, longer dsRNA may be cleaved prior to introduction into the cell, and/or Dicer may be activated at any time, thereby decreasing or eliminating the PKR response.

Suitable vectors include, without limitation, those described in U.S. Pat. Nos. 6,025,192, 5,888,732, 6,143,557, 6,171,861, 6,270,969, 5,766,891, 5,487,993, 5,827,657, 5,910,438, 6,180,407, 5,851,808 and PCT publications WO/9812339 and WO 00/01846 (all of which are incorporated in their entirety by this reference), which may be further modified according to the invention. Cloning of the sequence of interest can be achieved by enzymatic digestion of, for example, multiple cloning sites in the vector and ligation of the sequence of interest, which may be 100% identical to a region of the target gene, into the vector, or by other methods that will be apparent to one of ordinary skill in the art. A gene or sequence of interest may be inserted into vectors according to the invention by traditional cloning methods such as recombination technologies (Lox/Cre or Att), and other methods, which are well known in the art. Preferably, the sequence of interest is cloned into the vector by way of the Gateway cloning strategy as described in U.S. Pat. No. 6,143,557 and PCT WO8809372 (Karnaoukhova, et al. (2003) Construction of cDNA libraries by recombination using the CloneMiner™ cDNA library construction kit. Focus 25.2: 20-25.2) (all of which are incorporated in their entirety by this reference). Preferably, the vector includes a nucleotide sequence encoding a selectable marker including, but not limited to, markers that confer resistance to ampicillin, bleomycin, chloramphenicol, gentamycin, hygromycin, kanamycin, lincomycin, methotrexate, phosphinothricin, puromycin, carbenicillin, and tetracycline. In at least one embodiment of the invention, the nucleotide sequence encoding the sequence of interest is located between two promoters. The vector preferably contains an origin of replication to allow perpetual replication of the vector inside the organism. The vector most preferably contains a transcription termination sequence capable of stopping transcription at a specified cite on the template DNA.

Kanamycin selection may be utilized for easier production of recombinant plasmids according to the invention. For example, most genes to be transferred into the pTERMdT7 and pDONRdT7 vectors for dsRNA production exist in ampicillin resistant plasmids; therefore, kanamycin selection allows for recovery of only pTERMdT7 and pDONRdt7 with the gene of interest, without selection of the initial ampicillin resistant plasmid.

In certain embodiments, the vectors are episomal. In other embodiments, the vectors are chromosomally integrated. In either case, the sequence of interest may be transiently, conditionally or constitutively expressed. Further, chromosomally integrated vectors can produce a stably transformed or transfected cell line. Vectors for forming such stable cell lines include, without limitation, those described in U.S. Pat. No. 6,025,192 and PCT publication WO/9812339 (each of which is incorporated in its entirety by this reference).

Inducible promoters (tet, hormone receptors, and so on) may also be used to, for example, facilitate gene-silencing analyses by allowing the temporary suppression of normally lethal knockouts (e.g., “essential genes”) and aid in dissecting the sequential or temporal constraints of certain cellular phenomena. Furthermore, inducible vectors may be used, for example, to induce expression of the sequence of interest at a desirable time. For example, the sequence of interest may be under the control of a promoter derived from a gene upregulated in response to infection (e.g., Myb-type transcription factor, a late embryogenesis-abundant protein, a root-specific gene (i.e., TobRB7), D-ribulose 5-phosphate 3-epimerase, or a 20S proteasome α-subunit) by a pest, such as a member of platyhelminthes, thereby inducing expression of the dsRNA in response to infection.

Promoters are incorporated into the vector to initiate transcription. Suitable promoters include any nucleotide sequence capable of initiating transcription under appropriate conditions. Suitable promoters include, without limitation, pol III promoters; pol II promoters (see Paddison, P. J. et al., Stable suppression of gene expression by RNAi in mammalian cells, 99 Proc. Natl. Acad. Sci. U.S.A. 1443 (2002)), such as the Gal4 promoter, let858, SERCA, UL6, myo-2 or myo-3, Gal4p binding sites and/or Pho5; pol I promoters; viral promoters, such as T7, T3, and SP6, adenoviral promoters, the cytomegalovirus immediate early promoter, and the major operator and/or promoter regions of phage λ; yeast mating factor promoters (a or α); those disclosed in U.S. Pat. No. 6,537,786 (the contents of which are incorporated in their entirety by this reference), the polyhedron or p10 promoter of the baculovirus system and other sequences known to control the expression of genes and any combination thereof. A person of ordinary skill in the art may use any known or discovered promoter in combination with the invention. Promoters may be, for example, minimal, inducible, constitutive, tissue-specific, rheostatic, stress-responsive, or combinations thereof.

Preferably, an E. coli strain used to produce the dsRNA is an RNaseIII and even more preferably an RNase negative strain. Likewise, organisms and strains used to produce the dsRNA preferably have a depleted RNase activity.

The vector may contain one or more transcription terminators that stop transcription of the template DNA at a desired location. This may be used, for example, to limit transcription to the cloned sequence of interest and/or prevent transcription of vector DNA. Terminators may also be used to decrease the size of the product dsRNA to a size sufficient to reduce or eliminate the PKR response.

The term “transcription terminator” or “terminator” as used herein refers to a sequence signaling termination of transcription that is recognized by the polymerase, or a self-cleaving ribozyme (e.g., see Chowrira et al. 1994, J. Biol. Chem. 269: 25864), wherein a functional terminator sequence may be determined by incorporation into a primer extension template, wherein the terminator prevents the further extension of such primer extension product. The terminator may include a polyadenylation signal.

The exact length of a transcript is not generally critical and therefore a transcriptional terminator may be positioned at a wide range of positions relative the expressed nucleic acid and still have the desired effect of causing termination of transcription. Accordingly, a transcriptional terminator is operably linked to a transcribed nucleic acid provided that it mediates, or is compatible with, expression of the nucleic acid at a desired level. For example, a terminator operably linked to the sequence of interest should not cause premature termination (i.e. 3′ truncation) of the desired transcript and should function in the intended transcription source.

Suitable terminators include, without limitation, the T7, NusA, GTTE1 and GTTE2 (Carlomagno M S, Nappo A., NusA modulates intragenic termination by different pathways, 308 Genes 115 (2003)), λ NUT, λ tR2, Rho sites, tml, CaMV 35S, PI-II, TpsbA, Trps16, octopine (ocs) and nopaline synthase (nos) (Thornburg et al., Proc. Natl. Acad. Sci. USA 84:744,1987); An et al., Plant Cell 1:115 (1989), trp A, trp A (inverted), rrnBT1, rrnBT1 (inverted), rrnC, thr attenuator, phi 8-oop, OTH, lambda 65, lambda 65 (inverted) (Macdonald et al., (1993) Termination and slippage of bacteriophage T7 RNA polymerase J. Mol. Biol. 232(4): 1030-47) the pea rbcS E9 terminator and functional combinations thereof, see also U.S. Pat. Nos. 6,297,429, 6,518,066, 6,512,162, and 6,537,786 (all of which are incorporated in their entirety by this reference); Kashlev M, Komissarova N (2002) Transcription termination: primary intermediates and secondary adducts, J. Biol. Chem. 277(17):14501-8; Kakarin et al., (1998) Characterization of unusual sequence-specific termination signal for T7 RNA polymerase J. Biol. Chem. 273(30): 18802-11; Lyakhov et al., (1997) Mutant bacteriophage T7 RNA polymerases with altered termination properties, J. Mol. Biol. 269(1): 28-40; Lyakhov et al., (1998) Pausing and termination by bacteriophage T7 RNA polymerase, J. Mol. Bio. 280(2): 201-13; Macdonald et al., (1994) Characterization of two types of termination signal for bacteriophage T7 RNA polymerase, J. Mol. Biol. 238(2): 145-58; and Evgeny Nudler and Max E.Gottesman (2002) Transcription termination and anti-termination in E. coli. In addition, a gene's native transcription terminator may be used, Genes to Cells 7:755-768.

The invention allows for the generation of dsRNA at specific times of development and locations in an organism without introducing permanent mutations into the target genome. dsRNA and/or a vector capable of producing dsRNA may be directly introduced into the cell (i.e., intracellularly); or introduced extracellularly into a cavity, interstitial space, into the circulation of an organism, introduced orally, or may be introduced by bathing an organism in a solution containing dsRNA. Methods for oral introduction include direct consumption and adding or mixing dsRNA with food, which includes fluid intake, of the organism, as well as engineered approaches in which an organic material or species that is either consumed as food or capable of infecting the organism is engineered to express a dsRNA and then administered to the organism to be affected. For example, dsRNA may be transfected or transformed into a microorganism, such as a bacterial or yeast cell, which may then be fed to the organism. Physical methods of introducing nucleic acids are known in the art and include, but are not limited to, injection of a dsRNA solution directly into the cell or extracellular injection into the organism.

The invention further allows for the large-scale synthesis of siRNA using a biofactory, such as may be produced in bacteria or C. elegans. The biofactory organism may be engineered to produce dsRNA and fed to the target organism in which dsRNA inhibition is desired. The target organism may express, endogenously or by transgenesis, the gene one wishes to target. Since the target organism converts the dsRNA into large amounts of siRNA, this method can be used to generate large amounts of siRNA directed at a specific target gene. After a suitable period, allowing for optimal production of siRNA, the engineered biofactory organism may be delivered to a target organism. Alternately, the siRNA may be purified using standard molecular biological and chemical techniques before delivery to the target organism.

The dsRNA may include a siRNA or a hairpin, and may be transfected or transformed transiently or stably into a host.

The invention is useful in allowing the inhibition of essential genes. Such genes may be required for cell or organism viability at only particular stages of development or only in specific cellular compartments or tissues. The functional equivalent of a conditional mutation may be produced by inhibiting activity of the target gene under specified conditions or in a specific temporal, special or developmental manner. In certain embodiments, the target gene may be, without limitation, an endogenous gene of the target cell or organism, or a heterologous gene relative to the genome of the target cell or organism, such as a pathogen gene or gene introduced into a cell by recombination technologies

The cell having the target gene may be from the germ line or somatic, totipotent or pluripotent, dividing or non-dividing, parenchyma or epithelium, immortalized/transformed or primary, or the like. The cell may be a stem cell or a differentiated cell. Suitable cell types that are differentiated include, but are not limited to, adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

A eukaryotic target cell may be contained in or derived from, without limitation, animals; trypanosomes; plants including monocots, dicots and gymnosperms; fungi including both mold and yeast morphologies; or microbes including those used in agriculture or by industry, and those that are pathogenic for plants or animals.

Suitable plants include, without limitation, Arabidopsis; field crops (e.g., alfalfa, barley, bean, corn, cotton, flax, pea, rape, rice, rye, safflower, sorghum, soybean, sunflower, tobacco, and wheat); vegetable crops (e.g., asparagus, beet, broccoli, cabbage, carrot, cauliflower, celery, cucumber, eggplant, lettuce, onion, pepper, potato, pumpkin, radish, spinach, squash, taro, tomato, and zucchini); fruit and nut crops (e.g., almond, apple, apricot, banana, blackberry, blueberry, cacao, cherry, coconut, cranberry, date, fajoa, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon); and ornamentals (e.g., alder, ash, aspen, azalea, birch, boxwood, camellia, carnation, chrysanthemum, elm, fir, ivy, jasmine, juniper, oak, palm, poplar, pine, redwood, rhododendron, rose, and rubber).

Examples of suitable vertebrate animals include, e.g., fish and mammals (e.g., cattle, goat, pig, sheep, rodent, hamster, mouse, rat, primate, human, and puffer fish). Suitable invertebrate animals include, without limitation, nematodes, planaria, platyhelmithes, and other worms; Drosophila and other insects; and hydra. Representative genera of nematodes include those that infect animals (e.g., Ancylostoma, Ascaridia, Ascaris, Bunostomum, Caenorhabditis, Capillaria, Chabertia, Cooperia, Dictyocaulus, Haemonchus, Heterakis, Nematodirus, Oesophagostomum, Ostertagia, Oxyuris, Parascaris, Strongylus, Toxascaris, Trichuris, Trichostrongylus, Tflichonema, Toxocara, Uncinaria) and those that infect plants (e.g., Bursaphalenchus, Criconerriella, Diiylenchus, Ditylenchus, Globodera, Heicotylenchus, Heterodera, Longidorus, Melodoigyne, Nacobbus, Paratylenchus, Pratylenchus, Radopholus, Rotelynchus, Tylenchus, and Xiphinerna). Representative genera of platyhelmenthes that infect or attack animals include, without limitation, Arthurdendyus, Ascaris, Austroplana, Artioposthia, Bipallium, Dolichoplana, Geoplana, Schistosoma, Taenia and Trichuris. Representative orders of insects include Coleoptera, Diptera, Lepidoptera, and Homoptera.

Expression of the target gene is preferably attenuated so as to reproducibly produce a loss-of-function in the gene actively being targeted relative to a cell not exposed to dsRNA.

This and all aspects of the invention may be used to, inter alia, efficiently produce dsRNA; improve the strength of phenotypic expression; increase the number of individuals expressing the target phenotype; streamline the production of dsRNA-producing plasmids for a large number of genes; enhance production of dsRNA that is specific to the target gene by reducing or preventing transcription of the vector genetic backbone; and/or optimize the length of the dsRNA introduced in the cell. This and all aspects of the invention may be used in all organisms in which RNAi is effective, and in all applications that employ RNAi, including, but not limited to, genomic analysis (Clemens, J. C. et al. (2000) Use of double-stranded RNA interference in Drosophila cell lines to dissect signal transduction pathways, Proc. Natl. Acad. Sci. U.S.A. 97:6499-6503; Dobrosotskaya, I. Y. et al. (2002) Regulation of SREBP processing and membrane lipid production by phospholipids in Drosophila, Science 296:879-883), gene-silencing therapies, and drug development.

The invention also relates to a method of attenuating expression of a target gene in a eukaryotic cell, wherein dsRNA is introduced into the cell through a vector having at least one nucleotide sequence similar to the target gene, which, when transcribed, produces dsRNA in an amount sufficient to attenuate expression of the target gene.

In certain embodiments, transcription of the sequence of interest is initiated in both sense and antisense directions, wherein transcription from each strand is functionally linked to a transcriptional regulatory sequence, such as a promoter or enhancer, and a transcription terminator; where the transcriptional regulatory sequences initiate and terminate transcription in both directions, forming complementary transcripts; and where the complementary transcripts anneal to form the dsRNA. Where the formation of dsRNA is generated within a host cell, the complementary transcripts anneal under physiological conditions. In other embodiments, the vector may include two nucleotide sequences that, respectively, produce upon transcription two complementary sequences that anneal to form the dsRNA. In still other embodiments, the vector may include a nucleotide sequence that forms a hairpin upon transcription, where the hairpin forms an intramolecular dsRNA. In certain preferred embodiments, the vector transcribes the sequence of interest from both strands of the double helix, and may include at least one but preferably two transcription terminator sequences that cause transcription to stop.

Another aspect of the invention relates to a method of attenuating expression of a target gene in a eukaryotic cell, by introducing into a cell an vector having two promoters oriented such that, upon binding of an appropriate transcription factor to the promoters, the promoters are capable of initiating transcription of a sequence of interest located between the promoters, to generate dsRNA in an amount sufficient to attenuate expression of the target gene.

Yet another aspect of the invention relates to a method of attenuating expression of a target gene in a cell, by introducing into the cell a sequence of interest having a hairpin structure, in an amount sufficient to attenuate expression of the target gene, where the hairpin includes an inverted repeat of a nucleotide sequence that hybridizes under stringent conditions to the target gene. The hairpin nucleic acid may be, without limitation, RNA. The hairpin structure provides the dsRNA, thus, the sequence of interest may constitute a sequence derived from the mRNA of the target gene, a loop sequence and the complement of the mRNA sequence, such that a single transcription event will produce a dsRNA. Alternatively, the loop sequence may be a sequence recognized by an enzyme, such as a ribozyme.

Still another aspect of the invention relates to a method of identifying nucleic acid sequences responsible for conferring a particular phenotype in a cell. This method involves constructing a library of nucleic acid sequences from a cell in an orientation relative to a promoter to produce dsRNA; introducing the dsRNA library into a target cell; identifying members of the library which confer a particular phenotype on the cell; and identifying the nucleotide sequence corresponding to the library member which confers the particular phenotype. In this and all aspects of the invention, “corresponds to” includes, without limitation, being identical or homologous.

Therefore, there is provided a method of identifying DNA responsible for conferring a phenotype in a cell which comprises a) constructing a cDNA library or other library (e.g., a genomic library) of the DNA from a cell in a vector having at least two promoters capable of promoting transcription of the cDNA or DNA, which may include sequences flanking the cDNA or DNA, thereby producing dsRNA upon binding of an appropriate transcription factor to the promoters, b) having transcription terminator sequences operably linked to the cDNA or DNA sequence, c) introducing the library into one or more cells having the transcription factor, and d) identifying a desired phenotype of the cell having the desired library member and identifying, which may include isolating, the DNA or cDNA fragment from the library member responsible for conferring the phenotype. Optionally, the library may be organized into hierarchical pools, prior to step c) such as, for example, pools based on gene families. Likewise, known sequences can be studied using the described method, wherein the sequence of interest is inserted into the vector of step a) and carried through the method with appropriate modifications.

In yet an additional embodiment, the invention relates to a method of identifying a function of a gene in a planarian. The method involves producing a library of genes in a bacterial cell population, feeding the bacterial cell population to the planarian, and observing a change in a phenotype or a change at a cellular level. In one embodiment the planarian is S. mediterranea.

In another aspect, the invention relates to a method of screening for compounds that are involved in the pathogenesis of a cell. The method includes subjecting the cell to a stress, such as an infection, and altering gene expression in the cell using RNAi. The cell is observed for changes in phenotype or a change at the cellular level in response to the stress. For instance, in one embodiment, a eukaryotic cell is infected with a virus such as, for example Human Immunodeficiency Virus. RNAi is used to alter gene expression of the infected eukaryotic cell and a phenotype is assayed for such as, for example, determining if any eukaryotic cells live longer. The altered gene that causes a different phenotype, if present, in the eukaryotic cell is identified.

Yet another aspect of the invention relates to a method of conducting a drug discovery business. This method involves identifying by the subject assay a target gene that provides a phenotypically desirable response when inhibited by RNAi; identifying agents by their ability to inhibit expression of the target gene or the activity of an expression product of the target gene; conducting therapeutic profiling of agents identified in the immediately prior step, or further analogs thereof, for efficacy and toxicity in cells; and formulating a pharmaceutical preparation including one or more agents identified in the immediately prior step as having an acceptable therapeutic profile.

This aspect of the invention may include an additional step of establishing a distribution system for distributing the pharmaceutical preparation for sale, and may optionally include establishing a sales group for marketing the pharmaceutical preparation.

Another aspect of the invention relates to a method of conducting a target gene discovery business. This method involves identifying by the subject assay a target gene that provides a phenotypically desirable response when inhibited by RNAi; optionally conducting therapeutic profiling of the target gene for efficacy and toxicity in cells; optionally licensing, to a third party, the rights for further drug development of inhibitors of the target gene; and developing a drug to inhibit expression of the target gene.

The invention also relates to transgenic eukaryotes, which include a transgene encoding a dsRNA construct.

A eukaryote that is chimeric for the transgene is suitable in this aspect of the invention. In this and all aspects of the invention involving transgenes, the transgene may be located in one or more germline and/or somatic cells. The transgene may be, without limitation, chromosomally incorporated.

Suitable dsRNA constructs include, without limitation, constructs where the dsRNA is identical or similar to one or more target genes, preferably a target gene that is stably integrated into the genome of the cell in which it occurs. Also suitable are constructs that include a nucleotide sequence, which hybridizes under stringent conditions to a nucleotide sequence of a target gene; the sequence of interest may hybridize to, without limitation, a coding or a non-coding sequence of the target gene. “Similar nucleotide sequence” as used in this application means a first nucleotide sequence that hybridizes under stringent conditions to a target gene sequence complementary to the first nucleotide sequence.

Selectivity of hybridization exists when hybridization which is substantially more selective than a total lack of specificity occurs. Typically, selective hybridization will occur when there is at least about 70% homology over a stretch of at least about nine nucleotides, preferably at least about 85%, more preferably at least about 90%, and most preferably at least about 95%. The length of homology comparison may be over longer stretches, and in certain embodiments will often be over a stretch of at least about 14 nucleotides, usually at least about 20 nucleotides, more usually at least about 24 nucleotides, typically at least about 28 nucleotides, more typically at least about 32 nucleotides, and preferably at least about 36 or more nucleotides.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. The stringency conditions are also dependent on the length of the nucleic acid and the base composition of the nucleic acid, and can be determined by techniques well known in the art. For example, Asubel, 1992; Wetmur and Davidson, 1968.

Thus, as herein used, the term “stringent conditions” means hybridization will occur only if there is at least 85%, preferably at least 90%, more preferable 95% and most preferably at least 97% identity between the sequences. Such hybridization techniques are well known to those of skill in the art. Stringent hybridization conditions are as defined above or, alternatively, conditions under overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA, followed by washing the filters in about 0.1× to about 0.2×SSC at about 65° C. Hybridization techniques and procedures are well known to those skilled in the art and are described, for example, in Ausubel et al., Protocols in Molecular Biology, and Guide to Molecular Cloning Techniques.

dsRNA constructs may comprise one or more strands of polymerized ribonucleotide. The double-stranded structure may be formed by a single self-complementary RNA strand or two complementary RNA strands. RNA duplex formation may be initiated either inside or outside the cell. The dsRNA construct may be introduced in an amount, which allows delivery of at least one copy per cell. Higher doses of double-stranded material may yield more effective inhibition. Inhibition is sequence-specific in that nucleotide sequences corresponding to the duplex region of the RNA are targeted for genetic inhibition. In certain embodiments, dsRNA constructs containing a nucleotide sequences identical to a portion of the target gene are preferred for inhibition. RNA sequences with insertions, deletions, and point mutations relative to the target sequence have also been found to be effective for inhibition. Thus, sequence identity may be optimized by alignment algorithms known in the art and calculating the percent difference between the nucleotide sequences. Alternatively, the duplex region of the RNA may be defined functionally as a nucleotide sequence that is capable of hybridizing with a portion of the target gene sequence. In another embodiment, the dsRNA construct contains a nucleotide sequences identical to a non-coding portion of the target gene. Exemplary non-coding regions include, without limitation, introns, 5′ untranslated regions and 3′ untranslated regions. Sequences with insertions, deletions, and point mutations relative to the target non-coding sequence are also suitable.

Yet another aspect of the invention relates to a dsRNA for inhibiting expression of a mammalian gene. This dsRNA includes a first nucleotide sequence that hybridizes under stringent conditions to the target sequence or its compliment.

The sequence of interest may comprise, without limitation, at least 20 nucleotides, at least 25 nucleotides, at least 100 nucleotides, or at least 400 nucleotides. The sequence of interest may be substantially identical to, without limitation, at least one eukaryotic target gene, at least one coding sequence of at least one eukaryotic gene, and/or at least one non-coding sequence. The non-coding sequence according to this aspect may be nontranscribed, for example, when targeting RNA virus infectivity.

The sequence of interest may be capable of forming a hairpin structure having a first nucleotide sequence that hybridizes under stringent conditions to at least one mammalian gene; and a second nucleotide sequence which is a complementary inverted repeat of the first nucleotide sequence and hybridizes to the first nucleotide sequence to form a hairpin structure.

The dsRNAs may be designed to have a sequence that, for example, avoids highly conserved domain regions such as catalytic domains or ligand binding regions to circumvent inhibiting the translation of mRNAs of highly homologous multi-gene families; targets the 5′ and 3′ untranslated regions; accounts for any mRNA species potentially cross-reactive to the target mRNA; or will silence an entire class of targets. (Shuey, et al. (2002) RNAi: gene-silencing in therapeutic intervention, Drug Discovery Today 7(20):1040-1046).

Another aspect of the invention relates to a method of alleviating pest infestation of plants. This method involves identifying a DNA sequence of the pest that is critical for the pest's survival, growth, proliferation or reproduction; cloning the sequence or a fragment thereof into a vector capable of transcribing the pest sequence and its complement, thereby forming dsRNA, and introducing the vector into the plant under conditions effective to alleviate the pest infestation.

This aspect of the invention provides a selective mechanism for alleviating pest infestation. When the pest feeds on the plant, the dsRNA is taken up by cells in the pest, which digest the dsRNA. The digested dsRNA inhibits the expression of the identified pest sequence within the pest, which is critical for its growth, survival, proliferation, or reproduction, thus interfering with the pest's growth, survival, proliferation, or reproduction. This aspect of the invention is suitable for preventing, alleviating or treating pest infestation including, without limitation, nematode worms, insects, Tylenchulus ssp., Radopholus ssp., Rhadinaphelenchus ssp., Heterodera ssp., Rotylenchulus ssp., Pratylenchus ssp., Belonolaimus ssp., Canjanus ssp., Meloidogyne ssp., Globodera ssp., Nacobbus ssp., Ditylenchus ssp., Aphelenchoides ssp., Hirschmenniella ssp., Anguina ssp., Hoplolaimus ssp., Heliotylenchus ssp., Criconemellas ssp., Xiphinema ssp., Longidorus ssp., Trichondorus ssp., Paratrichondorus ssp., Aphelenchs ssp. and other plant pests. The dsRNA may be expressed in a specific plant tissue depending on the food source of the pest by using tissue specific promoters. Suitable plants include, without limitation, those listed above and any plant into which the dsRNA may be introduced. Preferably, the dsRNA is produced from a vector transcribing the sequence of interest and its complement and having transcription terminators located 3′ of the sequence of interest.

The invention further relates to a therapeutic method for alleviating parasitic helminth infestation of animals or humans: This method involves identifying a DNA sequence of the pest that is critical for the pest's survival, growth, proliferation or reproduction but preferably absent in the genome of the infected host; cloning the sequence or a fragment thereof into a vector capable of transcribing the sequence and its complement to produce dsRNA; and introducing the vector into the animal or human under conditions effective to alleviate the pest infestation.

The invention yet further relates to a method of alleviating the destruction of earthworm populations by helminthes. This method involves identifying a DNA sequence of the pest that is critical for the pest's survival, growth, proliferation or reproduction; cloning the sequence or a fragment thereof into a vector capable of transcribing the sequence and its complement to produce dsRNA; introducing the vector into earthworms under conditions effective to alleviate the pest infestation and placing these earthworms into areas where the earthworm population has been destroyed by or is under attack by helminthes.

The invention provides a selective mechanism for preventing, alleviating or treating pest infestation. When the pest infests the human or animal or feeds on the earthworm, the dsRNA is taken up by cells in the pest, which digest the dsRNA. The digested dsRNA inhibits the expression of the identified pest sequence within the pest, which is critical for its growth, survival, proliferation, or reproduction, thus interfering with the pest's growth, survival, proliferation, or reproduction. This aspect of the invention is suitable for preventing, alleviating or treating pest infestation including, without limitation, nematodes, platyhelmithes, Drosophila and other insects; and hydra. Representative genera of nematodes include those that infect animals (e.g., Ancylostorna, Ascaridia, Ascaris, Bunostomum, Caenorhabditis, Capillaria, Chabertia, Cooperia, Dictyocaulus, Haernonchus, Heterakis, Nematodirus, Oesophagostomum, Ostertagia, Oxyuris, Parascaris, Strongylus, Toxascaris, Trichuris, Trichostrongylus, Tflichonema, Toxocara, Uncinaria). Representative genera of platyhelmenthes that infect or attack animals include, without limitation, Arthurdendyus, Ascaris, Austroplana, Artioposthia, Bipallium, Dolichoplana, Geoplana, Schistosoma, Taenia and Trichuris. Representative orders of insects include Coleoptera, Diptera, Lepidoptera, and Homoptera. The dsRNA may be expressed in a specific tissue depending on the food source of the pest by using tissue specific promoters. Suitable animals include, without limitation, those listed above and any animal into which the dsRNA may be introduced. Preferably, the dsRNA is produced from a vector transcribing the sequence of interest and its complement and having transcription terminators located 3′ of the sequence of interest.

The invention also relates to the plasmid identified as pDONRdT7. In another aspect, the invention relates to a library of RNAi entry clones originating from a eukaryotic cell, such as a planarian, and further to methods of screening with the library. The library may be generated in a bacterial cell and introduced into the planarian by feeding.

Another aspect of the invention relates to a vector. This vector includes one or more promoters oriented relative to a DNA sequence such that the promoter is capable of initiating transcription of the DNA sequence to produce dsRNA. For example, the sequence of interest is cloned between attP1 and attP2 of pDONRdT7 or a similarly constructed vector.

In this and all aspects of the invention involving promoters, two promoters may flank the DNA sequence of interest. The DNA sequence, when not flanked by at least two promoters, may be in a proper sense orientation and in an antisense orientation relative to the promoter.

The invention also relates to a method of altering gene expression in an undifferentiated stem cell or the differentiated progeny thereof. The method involves introducing into the cell one or more dsRNAs according to the invention under conditions effective to alter gene expression in the stem cell or its progeny.

Suitable stem cells include, without limitation, embryonic stem cells and adult stem cells. Differentiated progeny include, without limitation, cells differentiated from embryonic stem cells and cells differentiated from adult stem cells.

Suitable embryonic stem cells are derived preferably from eukaryotes, more preferably from an animal. Embryonic stem cells may be isolated by methods known to one of skill in the art from, for example, the inner cell mass (ICM) of blastocyst stage embryos. Embryonic stem cells may, for example, be obtained from previously established cell lines or derived de novo by standard methods.

The embryonic stem cells may be the result of nuclear transfer. The donor nuclei may be obtained from, for example, any adult, fetal, or embryonic tissue by methods known in the art. In one embodiment, the donor nuclei are transferred to a previously modified recipient oocyte. Alternatively, the donor nuclei are modified prior to transfer.

In addition, the recipient oocyte may be modified prior to destruction of the oocyte nuclear material and transfer of the donor nuclei. Such a modification may be useful in preventing implantation of a zygote having the oocyte's nuclear complement. Mutations include, without limitation, any change in gene product or protein expression of an embryo derived from the modified oocyte, which prevents successful implantation in the uterine wall. Since implantation in the uterine wall is essential for fertilized mammalian embryos to progress beyond the blastocyst stage, embryos made from such modified oocytes could not give rise to viable organisms, thereby selecting for zygotes having the donor nuclear complement. Non-limiting examples of such modifications include those that decrease or eliminate the expression of a cell surface receptor required for the recognition between the blastocyst and the uterine wall; modifications that decrease or eliminate the expression of proteases required to digest the matrix in the uterine lining and thus allow proper implantation; and modifications that decrease or eliminate the expression of a protease necessary for the blastocyst to hatch from the zona pellucida where hatching is required for implantation.

The invention may be used to produce the phenotype of a “knock out” in such target genes as cell surface receptors, proteases, developmental genes (e.g., Hox genes), or any other target gene. For example, a Hox gene may be inserted into a vector similar to pDONRdT7 and introduced in an appropriate host cell. The host cell may be in the organism to receive the “knock out” or fed to the organism in which the “knock out” is desired, as appropriate. In another aspect, the target gene may originate from a library of genes obtained from a eukaryotic cell such as, for example, a library of genes from the planarian S. mediterranea.

A promoter sequence may be an inducible promoter or a functional fragment thereof, or other promoter sequence recognized in the dsRNA production system. A duplicate promoter may be inserted into the complementary sequence corresponding to a position 3′ of the first transcript that is to form the dsRNA, thereby producing promoters flanking the sequence of interest. Transcription termination sequences may be inserted outside of the flanking promoters. The construct may then be transfected into a cell, randomly integrated or additional sequences may be added to the vector to facilitate homologous recombination. Wherein the promoter is an inducible promoter, such as a heat shock promoter, the organism is subjected to an inducing event, such as heat shock, which produces the dsRNA, thereby inhibiting expression of the Hox gene in the organism.

Embryonic stem cells or embryonic stem cells obtained from fertilization of modified oocytes, or the differentiated progeny of the oocytes, can be further modified by introducing one or more additional dsRNAs into the cell.

Exemplary adult stem cells include, but are not limited to, hematopoietic stem cells, mesenchymal stem cells, cardiac stem cells, pancreatic stem cells, and neural stem cells. Exemplary adult stem cells include any stem cell capable of forming differentiated ectodermal, mesodermal, or endodermal derivatives. Non-limiting examples of differentiated cell types which arise from adult stem cells include blood, skeletal muscle, myocardium, endocardium, pericardium, bone, cartilage, tendon, ligament, connective tissue, adipose tissue, liver, pancreas, skin, neural tissue, lung, small intestine, large intestine, gall bladder, rectum, anus, bladder, female or male reproductive tract, genitals, and the linings of the body cavity.

Altering target gene expression includes, without limitation, alterations that decrease or eliminate Major Histocompatibility Complex (MHC) expression. Cells modified in this way will be tolerated by the recipient, thus avoiding complications arising from graft rejection. Such modified cells are suitable for transplantation into a related or unrelated patient to treat a condition characterized by cell damage or cell loss; and alterations that decrease or eliminate expression of genes required for viral or bacterial infection.

In another aspect, the RNAi methods of the present invention are used for a planarian RNA-mediated genetic interference (RNAi) screen, which introduces large-scale gene inhibition studies to this classic system. Planarians have been a classic model system for the study of regeneration, tissue homeostasis, and stem cell biology for over a century, but have not historically been accessible to extensive genetic manipulation. In one embodiment, 1065 genes of a planarian were screened. Phenotypes associated with the RNAi of 240 genes identify many paradigms for the study of gene function, and define the major categories of defects of planarians that display gene perturbations.

In an additional embodiment, the planarian may be screened for a phenotype with a heterologous gene from another organism. For instance, a library of human genes may be generated in a bacterial cell population, wherein the bacterial cell population including the human library is introduced into the planarian in order to screen for phenotypes or other cellular changes. In this manner, the function or effect of genes heterologous to the planarian may be studied.

In one embodiment, the effects of inhibiting genes with RNAi on tissue homeostasis in intact animals and neoblast proliferation were assessed in amputated animals, thus, identifying candidate stem cells, regeneration, and homeostasis regulators. The instant invention demonstrates the great potential of RNAi for the systematic exploration of gene function in understudied organisms and establishes planarians as a new and powerful model for the molecular genetic study of stem cells, regeneration, and tissue homeostasis.

Planarians are bilaterally symmetric metazoans renown for their regenerative capacities, extensive tissue turnover and regulation as part of their normal homeostasis, and the presence of a pluripotent adult stem cell population known as the neoblasts. These prominent attributes of normal planarian biology relate to classic problems of developmental biology and in vivo stem cell regulation that cannot be readily investigated in other commonly studied organisms^{1,2}.

Given these problems are poorly understood and are of importance to the life of most metazoans, in another embodiment, the genetic regulation of metazoans in the planarian Schmidtea mediterranea was explored. Large scale functional genetic surveys has been undertaken and pivotal in understanding the biology of multiple metazoans, including Drosophila melanogaster3, Caenorhabditis elegans⁴, and Danio rerio^{5,6}. However, such an approach has been precluded by planarian life cycles. The development of dsRNA-mediated genetic interference (RNAi)⁷ and the application of RNAi to systematic studies of gene function⁸⁻¹⁰ has opened the door for a new generation of genetic manipulations. In the instant invention, 1065 genes were selected as a representative sampling of the planarian S. mediterranea genome, a large-scale, RNAi-based screening strategy is disclosed to systematically disrupt their expression and assess their function in planarian biology. This screen defines the major phenotypic categories that exist in planarians following gene perturbation.

In embodiment, the method of screening the planarians includes, first, comparing regeneration phenotypes to defects observed in animals lacking neoblasts. Second, the method includes assessing differentiation and patterning within abnormal blastemas by antibody staining to understand the extent of new tissue formation and patterning that occurred. Third, it was determined whether proliferating neoblasts were present in appropriate numbers in animals that failed to regenerate and in newly amputated animals. Finally, genes important for regeneration and observed intact animals were inhibited to identify genes that regulate the homeostatic activities of neoblasts and those specifically involved in regeneration. The RNAi screening strategy utilizes the fact that the sequences of the genes perturbed are known, allowing for the association of phenotypes with predicted encoded biochemical function(s). The diverse phenotypes uncovered reveal the function for novel genes, identify previously unknown interactions between genes, and define novel roles for genes characterized in other organisms. The instant invention establishes novel paradigms for the exploration of how genes control metazoan biology, including regeneration and the in vivo regulation of stem cells.

Planarians are currently viewed as members of the Lophotrochozoa, which are one of the three major phyletic groupings of bilaterally symmetric animals²⁷. The other two groupings are known as the Ecdysozoa, which include C. elegans and Drosophila, and the Deuterostomes, which include the vertebrates. The Lophotrochozoa include a diverse set of animals such as mollusks, nemertean worms, and annelids that display a number of biological attributes not saliently manifested by current ecdysozoan model systems. The screen of the planarian S. mediterranea described herein, involving 1,065 genes and 53,400 amputations, is the first systematic loss of gene function study of any Lophotrochozoan and discovers defects associated with the RNAi of 240 genes that define the major planarian regeneration and homeostasis phenotypic categories. Many of these phenotypes involve aspects of metazoan biology that are prominent in planarians, but that cannot easily be studied in Drosophila and C. elegans, including, regeneration, adult pluripotent stem cells, and extensive tissue turnover as part of normal homeostasis. As such, the screen of the instant invention exemplifies the usefulness of such analyses in the Lophotrochozoa for informing the evolution of gene pathways and for investigating processes relevant to human development and health not easily studied in current invertebrate genetic systems.

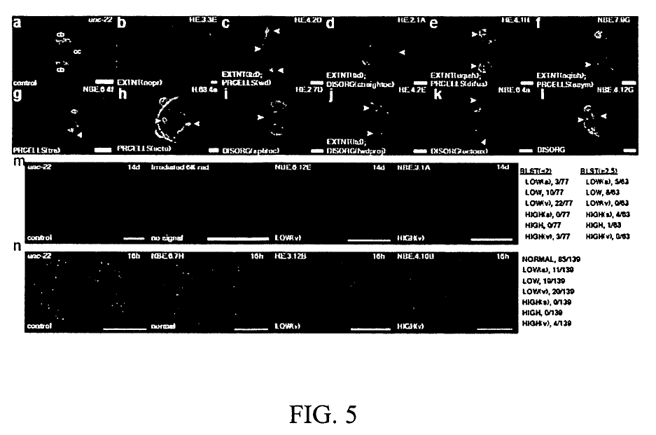

In a further aspect on the instant invention, the planarian phenotypes uncovered herein identify functions for novel genes and novel functional gene associations, as well as identify roles for genes characterized in other organisms in novel biological processes (FIG. 5E). For instance, the function of 35 novel genes and 38 human disease genes is ascribed herein, as well as defining experimental methods for functional studies of more. 85% of the genes associated with RNAi phenotypes are evolutionarily conserved. Thus, the roles for many conserved genes in understudied aspects of metazoan biology are ascribed herein. The instant invention discovered that there are multiple categories of regeneration-defective planarian phenotypes and discloses methods for distinguishing between them. One category appears to be needed for the functioning of neoblasts in regeneration since they resemble irradiated animals lacking neoblasts; i.e., inability to regenerate, curling, and lysis. Many genes associated with these RNAi-induced phenotype attributes, not surprisingly, are predicted to control basic cell functions (FIG. 5E). However, others appear to be more specific and encode, for example, an argonaute-like protein, other RNA-binding proteins, signal transduction proteins such as a phosphatidyl inositol transfer protein, chromatin regulators, and counterparts of two human disease genes as identified herein. These genes may be important for the functioning of stem cells in all animals. Some of these genes caused low numbers of neoblast mitoses following RNAi, indicating that they probably are required for basal neoblast functioning, whereas RNAi of others did not grossly affect neoblast mitoses, indicating they may be required for the functioning of neoblast progeny (FIG. 5E). Other genes are needed for regeneration, but did not cause curling or block neoblast mitoses following RNAi (FIG. 5E). These genes may function in blastema formation.

In yet an additional aspect, the present inventions discovers genes that are needed for regeneration, but are not needed for homeostasis or do not cause tissue regression or curling in intact animals following RNAi. These genes may control regeneration initiation, blastema formation, and the differentiation of neoblast progeny (FIG. 5E). For example, since a gene encoding a SMAD4-like protein is dispensable for neoblast function in homeostasis but is needed for regeneration, TGF-β signaling may control the initiation of planarian regeneration.

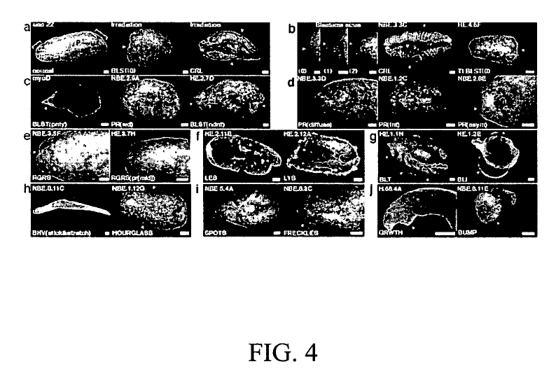

The instant invention also discloses that all genes critical for homeostasis are not needed for regeneration or neoblast proliferation, suggesting homeostasis involves both neoblast control of cell turnover as well as the regulated patterning and functioning of differentiated tissues (FIG. 5E). This observation is supported by the fact that adult planarians are constantly regulating the size and scale of their various organ systems²⁸ and by the observation that some homeostasis defects involved the formation of lesions in the shape of underlying organs (FIG. 4F). Numerous other striking phenotypes were uncovered, involving, for example, abnormal behavior, lesions, growths, asymmetry, abnormal patterning, abnormal posture, defective caudal blastema formation, and abnormal pigmentation. These phenotypes identify genes that control the patterning of blastemas and the functioning of regenerated animals, ascribe functions for many genes, and define many paradigms for the exploration of planarian biology.

In another embodiment, the RNAi screen of the instant invention demonstrates the use of RNAi to perform large-scale functional analyses of genes in non-standard genetic organisms that require primarily a characterized cDNA collection and appropriate animal culture and dsRNA delivery methods. Such analyses are of major importance for the study of the evolution of genes and their functions, and for the exploration of understudied, conserved biological processes in animals. One discovery of the instant invention establishes that S. mediterranea as an effective organism for the study of genes involved in disease, stem cells, homeostasis, and regeneration.

The invention disclosed herein may be more readily understood by reference to the following examples, which are included merely for purposes of illustration of certain aspects and embodiments of the invention and are not intended to limit the invention.

### EXAMPLES

### Example 1

### Construction of the Plasmid Vector pDONRdT7

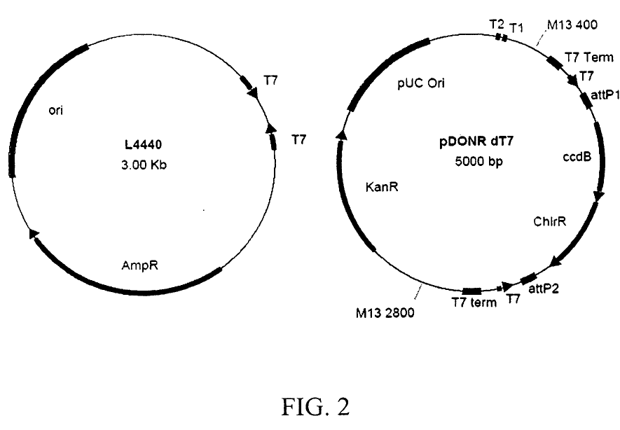

The RNAi vector pDONRdT7, shown in FIG. 2, was constructed for the generation of an S. mediterranea RNAi library. L4440, also shown in FIG. 2, is the standard vector used for feeding bacteria that express dsRNA to C. elegans and has been successfully used for a C. elegans RNAi screen. Two opposing T7 promoters are incorporated that allow for the production of dsRNA. To improve dsRNA production, T7 terminators were utilized to ensure that transcription from the T7 promoters generates only dsRNA from the cDNA insert and not the vector. The current S. mediterranea cDNAs are in a Bluescript vector. These cDNAs can be amplified by PCR using primers that recognize the vector sequence and contain att recombination sequences, and that recombine with the att recombination sites in pDONRdT7 in a single one hour reaction on the bench top. This strategy utilizes the replacement of a toxic ccdB gene with the cDNA for selection in bacteria, and is a modified version of the Gateway® (Invitrogen™) gene cloning strategy. pDONRdT7 has been successfully constructed and used for the transfer of cDNAs.

### Example 2

### RNAi of C. elegans unc-22 Gene

RNAi of the C. elegans gene unc-22 results in a twitching phenotype in adult C. elegans. The unc-22 cDNA was transferred into vector pDONRdT7 using a Gateway recombination reaction (Invitrogen™), and pDONRdT7 was found to be more effective for RNAi than the original L4440 vector, as shown in Table 1, below. pDONRdT7 allows for the efficient cloning of a large number of cDNAs, generally more effective than existing art, and works with 100% efficiency to generate RNAi phenotypes in planarians in this example.

TABLE 1
pDONR dT7 is effective for RNAi in C. elegans

| | dsRNA induction | dsRNA induction |
|---|---|---|
| construct | in liquid | on plates |
| L4440 unc-22 | 26% (9/35) | 74% (61/82) |
| pDONRdT7 unc-22 | 96% (43/45) | 100% (129/129) |

### Example 3

### RNAi of Planarian PC2 Gene

PC2 is the planarian pro-hormone convertase 2 gene and is required for proper locomotion. PC2 was transferred into pDONRdT7 using a Gateway® recombination reaction (Invitrogen™), used to produce dsRNA of PC2 in bacteria, which were then mixed with food suitable for planarians (liver homogenate) and fed to the planarians once per day for either one, two or three consecutive days. In all cases, 100% of the subject animals demonstrated a locomotion phenotype after a single round of feeding, as shown in Table 2, below.

TABLE 2
pDONR dT7 works for RNAi by feeding in planarians

| | % immobilized | | |
|---|---|---|---|
| | One round of | Two rounds of | |
| construct | RNAi feeding | RNAi feeding | RNA injection |
| pDONRdT7 PC2 | 100% (20/20) | 100% (20/20) | 100% (20/20) |

Thus, the inclusion of transcriptional terminator sequences for both transcripts of the dsRNA results in an increase in efficiency of inhibition. One possible cause of this new and unexpected result is believed to be due to restricting transcription to the cloned cDNA. Without the presence of flanking transcriptional terminators, transcription proceeds into the vector resulting in the production of a large RNA transcript coding the cloned cDNA as well as the vector DNA sequence. The terminators help ensure that only the cloned cDNA is transcribed, thus, increasing the yield of double stranded RNA molecules effecting gene-specific RNA-mediated genetic interference.

### Example 4

### Construction of RNAi Library

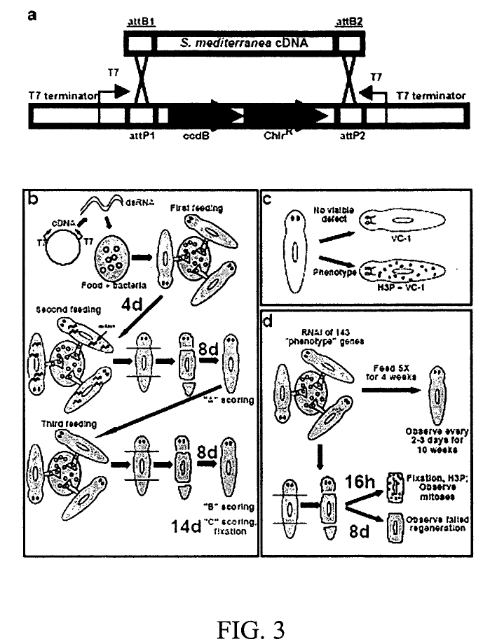

pDONRdT7 was generated by creating a PCR fragment from L4440¹³ that contained two T7 promoter sequences flanking the L4440 multiple cloning site region, two class I T7 terminators, and StuI and AflII restriction sites. This fragment was cloned into pDONR221 (Invitrogen) at the AflII/EcoRV restriction sites. An ApaI/EcoRV fragment from pDONR221, containing the attP recombination sequences, a chloramphenicol resistance gene, and the toxic mutant ccdB gene, was cloned into the ApaI/SmaI sites on the resultant plasmid. To generate RNAi library clones, cDNAs within a pBluescript vector from neoblast-enriched and head libraries²⁹ were PCR amplified individually using primers that recognize pBluescript and contain attB recombination sequences. PCR products were individually cloned into pDONRdT7 using a BP reaction (Invitrogen) to create RNAi entry clones (FIG. 3A). RNAi entry clones were individually transformed into the E. coli strain HT115¹³for RNAi.

### Example 5

### RNAi

Bacteria containing RNAi clones were grown overnight in 2xYT media containing kanamycin and tetracycline. Overnight cultures were diluted 1:10 in fresh media, grown to OD 0.4 at 37° C., and induced with 100 mM IPTG for 2 hours (h). To feed 10 animals, 2.5 mL of bacteria were collected by centrifugation and resuspended in 25 μL 1:1 homogenized liver (previously blended and passed through stainless steel mesh):water. This suspension was mixed with 9.4 μL 2% ultra-low gelling temperature agarose and 0.7 μL red food coloring and allowed to solidify on ice in ˜10 μL spots. Room temperature (RT) RNAi food was fed to planarians. After four days, animals were fed the RNAi food for a particular gene again, and 3.5 hours after this feeding, the heads and the tails removed with a scalpel. After nine days of regeneration the animals were fed and amputated again (FIG. 3B). For assessing tissue homeostasis in RNAi animals, four feedings were performed. Some of the genes from the pilot screen were inhibited by injecting dsRNA 3×32 nL on three consecutive days, amputating, injecting 3×32 nL following regeneration, and amputating again. The asexual clonal CIW4 line of S. mediterranea animals were used for these studies and maintained as previously described¹⁹.

### Example 6

### Antibody Labeling

Animals were killed in 4° C. 2N HCl for five minutes, fixed in Carnoy's fixative (60% ethanol, 30% chloroform, 10% glacial acetic acid) for 2 hours on ice, placed in methanol at −20° C. for 1 hour, and bleached overnight in the light at RT in 6% hydrogen peroxide in methanol. Animals were rinsed two times in methanol and stored at −20° C. Following rehydration in a 75%, 50%, 25% MeOH:PBTx (PBS+0.3% triton x-100) series, and two rinses in PBTx, animals were blocked for 6 hours at RT in PBTxB (PBTx+0.25% BSA) or PBTxBH (PBTxB+10% horse serum). Throughout the procedure animals were maintained at room temperature (RT), rocking. Animals were incubated overnight with 1:5000 α-phosphorylated histone H3 (kind gift of Dr. C. A. Mizzen), 1:5000 α-arrestin (kind gift of Dr. K. Agata), and/or 1:133 α-synaptotagmin (kind gift of Dr. K. Agata). Labeled animals were rinsed for 5 minutes in PBTxB, then 1× per hour, 6 times. Animals were labeled overnight in 1:400 goat α-mouse Alexa488 (Molecular Probes) or in 1:100 goat α-rabbit-HRP (Molecular Probes). Animals were washed for 5 minutes, then 1× per hour, 6 times. For those labeled with α-mouse-488, animals were mounted in Vectashield (Vector). For those labeled with α-rabbit-HRP, animals were incubated with 1:100 tyramide-Alexa568 in amplification buffer (Molecular Probes) for one hour. Animals were rinsed 5× for 5 minutes each in PBTxB, then 4× for 30 minutes, each in PBTxB. Animals were stored overnight in the dark at 4° C. The animals were rinsed 6× for 1 hour each at RT and mounted in Vectashield (Vector).

### Example 7

### RNAi Screen in S. Mediterranea

RNAi has been demonstrated to disrupt expression of S. mediterranea genes with a high degree of efficiency and specificity^{11,12}. The RNAi by feeding methodology used for the screen of the instant invention, involves expressing dsRNA from a planarian gene in bacteria and suspending those bacteria with the commonly used planarian food of blended liver, mixed with agarose¹². The effectiveness of the feeding method and protocol used in this manuscript (FIG. 3, methods) was maximized through extensive optimization experiments (data not shown). An RNAi vector (pDONRdT7) was generated that contains two T7 RNA polymerase promoters flanked by two class I T7 transcriptional terminators and that utilizes a modified Gateway cloning strategy (Invitrogen) to facilitate cDNA transfer (FIG. 3A). The data of the instant invention indicates that the presence of T7 terminators in this vector results in more effective RNAi than that seen with conventional vectors¹³ in C. elegans and planarians (data not shown).

S. mediterranea cDNAs randomly selected from two cDNA libraries were inserted into pDONRdT7 and introduced into the RNaseIII-deficient bacterial strain HT115¹³. 1065 of these genes were inhibited using RNAi by feeding (FIG. 3B). For each gene to be inhibited, the dsRNA food was fed to planarians twice in the span of five days. The heads and tails of eight planarians per gene were surgically removed and following eight days of regeneration, animals were scored for defects (“A” scoring) (FIG. 3B). On the following day, animals were fed the dsRNA food again and the regenerated heads and tails were surgically removed. After another eight days, the animals were scored (“B” scoring) for the size of the head blastemas on trunks and tails, the size of the tail blastema on heads, the ability of tails to regenerate a pharynx in the pre-existing tissue, the shape of the blastemas, the presence and pattern of photoreceptors, light response, vibration response, touch response, flipping, locomotion, turning, and head lifting. Following another six days, animals were scored for changes in any pre-existing phenotype or for the development of a new defect (“C” scoring). Many animals, both with and without a detectable defect, were fixed and analyzed by antibody labeling to detect additional phenotypes or defects at the cellular level (FIG. 3C). Multiple RNAi feedings and two rounds of regeneration helped to minimize protein perdurance. The A, B, and C scoring timepoints served to determine different degrees of phenotype expressivity, since aspects of a particular gene phenotype might be observed in the A scoring and precluded by a more severe aspect of the phenotype in the B scoring.

### Example 8

### Identification of Multiple New Paradigms for the Study of Gene Function

The types of phenotypes that would be uncovered by affecting gene function in planarians were unknown. Of the 1065 genes perturbed by RNAi, 240 (22.5%) conferred specific phenotypes when perturbed (Table 3 and Tables 4 and 5 of the incorporated PCT International Application PCT/US2004/037475 (corresponding to PCT International Publication WO 2005/047300 A2 of May. 26, 2005)). A sampling of the spectrum of phenotypes observed can be found in Table 3 and FIGS. 4A-J. The major phenotypic categories uncovered include the inability to regenerate (FIG. 4B), curling of animals around their ventral surface (FIG. 4B), blastema shape and morphology abnormalities (FIG. 4C), a variety of photoreceptor abnormalities (FIG. 4D), behavioral defects (Table 3 and Table 4 of the incorporated PCT International Application PCT/US2004/037475 (corresponding to PCT International Publication WO 2005/047300 A2 of May. 26, 2005)), tissue regression (FIG. 4E), lesions (FIG. 4F), and lysis (FIG. 4F). Regeneration-abnormal phenotypes have been categorized using a nomenclature system described in Table 4 of the incorporated PCT International Application PCT/US2004/037475 (corresponding to PCT International Publication WO 2005/047300 A2 of May 26, 2005). A large number of unexpected and surprising phenotypic categories were also uncovered (Table 4). Examples include defects unique to caudal blastemas (TLBLST) when perturbed (Table 4, FIG. 4B), animals that glide sideways (Table 4), animals with signs of asymmetry (Tables 4, 5, FIGS. 4D, 5F), animals with abnormal posture (FIG. 4H), animals with pigment “freckles” in the normally unpigmented blastemas or body spots (FIG. 4I), and animals with ectopic growths and photoreceptors (FIGS. 4J, 5H). These novel phenotypes establish unique paradigms to study the genetic control of diverse aspects of the poorly understood biology of planarians.

### Example 9

### S. mediterranea Genes Associated With RNAi Phenotypes are Conserved

Of the 240 genes associated with RNAi phenotypes, 205 (85%) are predicted to encode proteins with significant homology to those encoded in the genomes of other organisms (Tables 4, 5 of the incorporated PCT International Application PCT/US2004/037475 (corresponding to PCT International Publication WO 2005/047300 A2 of May 26, 2005)). This high frequency, coupled with the diverse set of predicted functions for these genes (Table 3), demonstrates the utility of studies of S. mediterranea for broadly informing general metazoan biology. For example, 38 of the identified genes associated with RNAi phenotypes are related to human disease genes (Table 6). These genes cause an array of phenotypes; for example, ranging from aberrant regeneration following RNAi of a spastic paraplegia gene¹⁴ to aberrant photoreceptor regeneration and functioning following RNAi of an RGS9-like encoding gene, which is associated with bradyopsia in humans¹⁵.

Given that only eight of these 38 genes have a corresponding mouse knockout model, the phenotypes observed in S. mediterranea provide new information on the functions of disease genes and demonstrate the utility of S. mediterranea for the study of orthologs of human genes involved in genetic disorders. Moreover, the remaining 35 genes associated with RNAi phenotypes, for which no obvious homologues were found in other phyla, may also be of medical relevance. These genes may be specific to the Platyhelminthes and might, thus, be required for the survival of their related pathogenic brethren, the cestodes and trematodes (Tables 4, 5 of the incorporated PCT International Application PCT/US2004/037475 (corresponding to PCT International Publication WO 2005/047300 A2 of May 26, 2005)). Considering such pathogens are estimated to cause disease in nearly 300 million people throughout the world, these genes may make attractive drug targets. Together, the instant invention provides insights into the functioning of a large number of genes that control metazoan biology, as well as present new information on the functions of disease genes.

### Example 10

### Similar Phenotypes Identify Genes With Shared Functional Activities

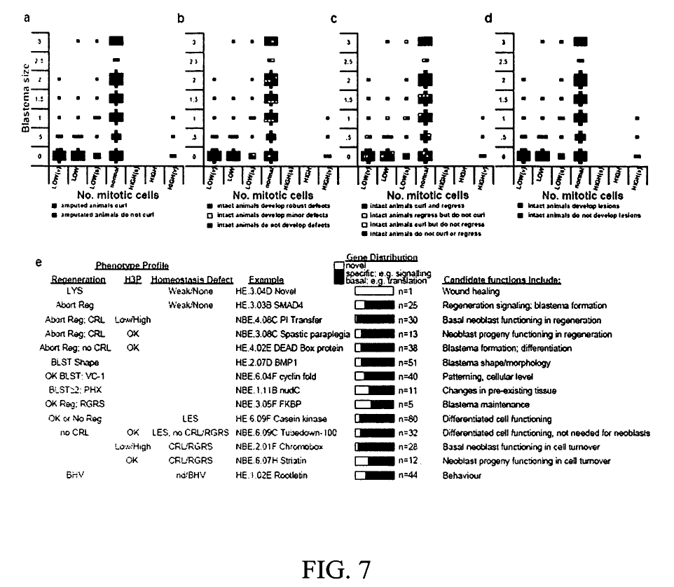

In multiple cases, genes predicted to act together confer similar phenotypes when perturbed independently by RNAi. For instance, RNAi of two genes that encode different subunits of the ARP2/3 complex with very different nucleotide sequences (HE.2.11E, HE.2.12A) caused early lysis; RNAi of two genes encoding components of TGF-β signalling (HE.2.07D, HE.3.03B) caused indented blastemas; and RNAi of α and β-tubulin-encoding genes (HE.1.03G, HE.1.01H) caused uncoordinated behavior, blisters, and bloating (Tables 4 and 5 of the incorporated PCT International Application PCT/US2004/037475 (corresponding to PCT International Publication WO 2005/047300 A2 of May 26, 2005)). These data demonstrate the ability of S. mediterranea RNAi studies to identify multiple pathway components involved in diverse biological events. These observations also indicate that the screen of the instant invention provides predictive power, i.e., genes with unknown function or unknown association to other genes may act together with those genes that share a similar RNAi phenotype. For instance, RNAi of NBE.3.07F or NBE.5.04A caused spots, blisters, and bloating (FIG. 4I). The first is similar to hunchback and the other encodes a POU domain protein (Table 4 of the incorporated PCT International Application PCT/US2004/037475 (corresponding to PCT International Publication WO 2005/047300 A2 of May 26, 2005)). Thus, these two transcription factors may act together. Also, RNAi of HE.1.08G or NBE.8.03C caused freckles (FIG. 4I). The first encodes an α-spectrin-like protein and the other encodes a protein with no known predicted function that may thus act with α-spectrin (Table 4). For the many other phenotypic categories, including regeneration and neoblast abnormalities, the data in Tables 4, 5, 7, 8, and FIGS. 7A-E identify shared properties that point to many candidate functional associations (see below).

### Example 11

### Proliferation and Patterning Phenotypes at the Cellular Level

In order to determine the frequency with which cellular defects occurred but did not cause a phenotype detectable with light microscopy, the photoreceptor neurons of animals with no visible phenotype were labeled with an anti-arrestin antibody (VC-1)¹⁶. Animals from the RNAi of 564 genes were tested. The photoreceptor neurons were chosen for study because they are easy to score for defects¹², exist in two well-defined clusters of 24 cells each and extend easily visualized posterior and ventral processes to the cephalic ganglia^{17,18} (FIG. 5A). Ten new genes associated with cellular phenotypes following RNAi were identified in this manner (Tables 5, 7 of the incorporated PCT International Application PCT/US2004/037475 (corresponding to PCT International Publication WO 2005/047300 A2 of May 26, 2005). Additional animals with indeterminate morphological defects resulting from the RNAi of another 113 genes were also labeled with VC-1. These animals were utilized to ascertain the feasibility of detecting proliferation defects by labeling with an antibody that recognizes mitotic neoblasts¹⁹ (αH3P, anti-phosphorylated histone H3²⁰). Analysis of the αH3P data required first quantifying the number of mitotic cells in control animals (Table 7 of the incorporated PCT International Application PCT/US2004/037475 (corresponding to PCT International Publication WO 2005/047300 A2 of May 26, 2005), FIG. 5M) and categorizing differences from the control in the mitotic numbers found in RNAi animals. 10 additional genes were determined to be associated with proliferation defects following RNAi and three of these also had photoreceptor neuron abnormalities (Table 7 of the incorporated PCT International Application PCT/US2004/037475 (corresponding to PCT International Publication WO 2005/047300 A2 of May 26, 2005)).

Next, animals that regenerated abnormally were used from the RNAi of 140 genes, and defects in patterning, differentiation, and neoblast proliferation were assessed by fixing specimens after 14 days of regeneration and labeling them with VC-1 and a αH3P. Analysis of the VC-1 data uncovered a large variety of photoreceptor abnormalities (Table 7 of the incorporated PCT International Application PCT/US2004/037475 (corresponding to PCT International Publication WO 2005/047300 A2 of May 26, 2005), FIG. 5B-L). Phenotypes include limited regeneration of the photoreceptor system (FIG. 5B-F), photoreceptor cell bodies dispersed posteriorly from the main neuron cluster (“tears” phenotype) and/or ectopic photoreceptors (FIGS. 5G, H), diffuse clusters of photoreceptor neurons (FIG. 5E), asymmetric photoreceptor cell body clusters (FIG. 5F), optic chiasmata defects (FIG. 5D, I), axon abnormalities (FIGS. 5J, K), and general disorganization (FIG. 5L). These defects reveal cellular and patterning abnormalities associated with specific gene perturbations that could not have been observed by light microscopy (Table 7 of the incorporated PCT International Application PCT/US2004/037475 (corresponding to PCT International Publication WO 2005/047300 A2 of May 26, 2005)).

Homologies of genes associated with these RNAi-induced patterning defects can be found in Table 7 (of the incorporated PCT International Application PCT/US2004/037475 (corresponding to PCT International Publication WO 2005/047300 A2 of May 26, 2005). Analysis of the αH3P data uncovered proliferation defects (Id., FIG. 5M). Of the 140 genes in this dataset, RNAi of 48 of the genes led to low mitotic cell numbers suggesting that their perturbation may cause abnormalities due to neoblast absence or inability to proliferate. A large majority of animals with lower than normal numbers of mitotic cells also had defects in the production of normal sized blastemas (Id.) RNAi of eight genes led to abnormally high numbers of mitotic neoblasts as compared to the control, indicating these animals may have developed regeneration abnormalities due to mitotic defects or misregulation of the neoblast population (Id.) RNAi of 84 genes led to relatively normal numbers of mitotic cells, indicating these animals may have developed defects due to dysfunction of cells other than neoblasts (Id.) These results identify distinct functional categories for all the genes tested and demonstrate the feasibility of performing screens for specific cellular defects in S. mediterranea (Table 7 of the incorporated PCT International Application PCT/US2004/037475 (corresponding to PCT International Publication WO 2005/047300 A2 of May 26, 2005)).

### Example 12

### Genes for Regeneration

Many genes important for regeneration have been identified. Blastema size abnormalities have been categorized on a scale from 0 to 3, with “BLST(0)” referring to no regeneration and “BLST(3)” referring to normal regeneration (FIG. 4B). However, a large number of defects not specific to regeneration, but affecting more general cellular processes may underlie the inability of animals to regenerate following RNAi. Because neoblasts are essential for regeneration to occur in planarians, two categories of experiments were designed to determine if genes important for regeneration were also important for the survival and proliferation of neoblasts. First, RNAi phenotypes were compared to those observed in animals lacking neoblasts. Second, genes needed for the production of normal-sized blastemas were inhibited and fixed animals after amputation to determine numbers of mitotic neoblasts during the initiation of regeneration.

Genes needed for tile functioning of neoblasts. Irradiation of planarians is known to specifically kill the neoblasts, block regeneration, and result in lethality²¹⁻²³. Irradiated (e.g., 6000 rad) and amputated wild-type S. mediterranea animals were observed to be incapable of regenerating (FIG. 4A), curled their bodies around their ventral surface within 15 days (FIG. 4A), and subsequently died by lysis. Therefore, genes that cause similar defects following RNAi may be needed for neoblast function in regeneration. 140 gene perturbations blocked, limited, or reduced regeneration (Table 3 herein, and Tables 4 and 5 of the incorporated PCT International Application PCT/US2004/037475 (corresponding to PCT International Publication WO 2005/047300 A2 of May 26, 2005)). The RNAi of 47 genes caused body curling around the ventral surface (CRL), similar to that seen in irradiated animals (Table 4 of the incorporated PCT International Application PCT/US2004/037475 (corresponding to PCT International Publication WO 2005/047300 A2 of May 26, 2005), FIG. 4B). Lysis was the typical fate of these curled animals (Table 4 of the incorporated PCT International Application PCT/US2004/037475 (corresponding to PCT International Publication WO 2005/047300 A2 of May 26, 2005)). These candidate stem cell regulatory genes for regeneration include, amongst anticipated basal cell machinery factors, RNA binding proteins (HB.14.6D, NBE.4.06D, NBE.7.07D, NBE.8.12D), signal transduction factors (NBE.4.08C, phosphatidyl inositol transfer protein), chromatin regulators (HE.2.01H, histone deacetylase), and disease genes (NBE.3.11F, chondrosarcoma-associated protein 2 and NBE.3.08C, human spastic paraplegia protein) (Tables 4, 5 of the incorporated PCT International Application PCT/US2004/037475 (corresponding to PCT International Publication WO 2005/047300 A2 of May 26, 2005)).

No labeling of irradiated animals was observed with αH3P, indicating αH3P specifically labels mitotic neoblasts (FIG. 5M). One hundred thirty-nine genes associated with RNAi phenotypes were inhibited and the resultant animals labeled 16 or 24 hours following amputation with αH3P. Fifty out of the 139 genes studied caused lower than normal numbers of mitotic cells following RNAi and amputation (Table 8, FIGS. 5N, 7A-D). The majority of these genes also perturbed the ability to regenerate following RNAi (FIGS. 7A-D). These genes might be important for neoblast maintenance or deployment. Four genes that cause very high numbers of mitotic cells following RNAi and amputation include two components of the proteasome, gamma tubulin, and CDC23 (subunit of anaphase promoting complex), indicating possible defects in chromosome separation at mitosis (Table 8 of the incorporated PCT International Application PCT/US2004/037475 (corresponding to PCT International Publication WO 2005/047300 A2 of May 26, 2005)). This hypothesis is supported by the observation that NBE.5.01A RNAi (another anaphase promoting complex subunit) screened animals also had high numbers of αH3P-labeled cells 14 days after amputation (Table 7 of the incorporated PCT International Application PCT/US2004/037475 (corresponding to PCT International Publication WO 2005/047300 A2 of May 26, 2005)). Genes that cause the ventral curling phenotype following RNAi and amputation are very likely to be required for regeneration (P<0.0001) and are often, but not always, associated with reduced mitotic cell numbers following amputation (FIG. 7A). That RNAi of genes can block regeneration, cause curling, and not reduce mitotic numbers suggest that postmitotic disruptions of neoblast function have also been identified (n=13 genes, Table 8 of the incorporated PCT International Application PCT/US2004/037475 (corresponding to PCT International Publication WO 2005/047300 A2 of May 26, 2005), FIG. 7E).

Genes necessary for regeneration but not for neoblast functionling. For 85 out of 139 genes inhibited by RNAi, animals that were labeled with αH3P following amputation had normal numbers of mitotic neoblasts (Table 8 of the incorporated PCT International Application PCT/US2004/037475 (corresponding to PCT International Publication WO 2005/047300 A2 of May 26, 2005), FIG. 5N). This observation does not exclude the possibility of subtle mitotic defects or other defects in the cell cycle. RNAi of 38 of these genes caused regeneration of very small blastemas (BLST<1.5, FIGS. 7A-E). These 38 genes represent a strikingly different set of gene functions from those genes needed for regeneration and that cause curling following RNAi or those that reduce numbers of mitotic cells. For example, of 30 genes that caused curling and abnormal numbers of mitotic neoblasts following RNAi and amputation, 18 encode ribosomal proteins and only one encodes a metabolic protein (Table 8 of the incorporated PCT International Application PCT/US2004/037475 (corresponding to PCT International Publication WO 2005/047300 A2 of May 26, 2005)). By contrast, of the 38 genes that were important for regeneration but not for neoblast mitoses, only one encodes a cytosolic ribosome component and eight are predicted to be involved in metabolism (Id.) Of the 38 genes important for regeneration but not needed for the presence and/or division of neoblasts, five are predicted to encode RNA-binding proteins (HE.1.07A, HE.2.01A, HE.2.09A, HE.2.09G, HE.4.02E) and five to encode signal transduction proteins (HE.3.03B, HE.4.05E, NBE.3.03G, NBE.4.12G, NBE.6.07H, Tables 4 and 8 of the incorporated PCT International Application PCT/US2004/037475 (corresponding to PCT International Publication WO 2005/047300 A2 of May 26, 2005)). These genes may control regeneration initiation, the ability of neoblast progeny to form differentiated cells or to organize to form a blastema.

### Example 13

### Tissue Homeostasis Defects Categorize Regeneration Gene Functions

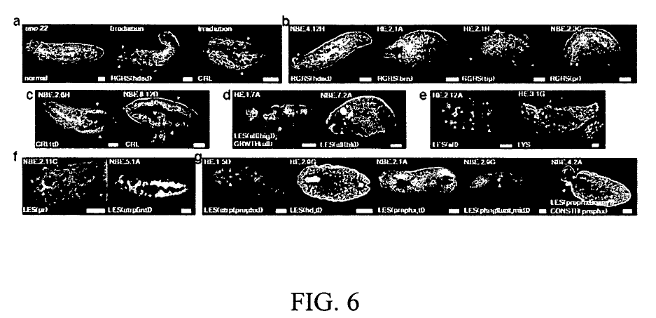

To further understand the cellular functions of genes required for regeneration, their function was studied in tissue homeostasis. Because neoblasts control the extensive cell turnover that occurs during normal adult planarian life¹⁹, observation of non-amputated, RNAi-treated animals allows an assessment of whether genes are required for all neoblast functions, have primary functions in regeneration, or are required for the functioning of differentiated cells. Using the knowledge of phenotypes from the regeneration screen herein, 123 genes were selected to represent a distribution of blastema-size phenotypes ranging from 0.5 to 2.5, following RNAi (Table 8 of the incorporated PCT International Application PCT/US2004/037475 (corresponding to PCT International Publication WO 2005/047300 A2 of May 26, 2005)). Another 20 genes were selected that cause a variety of other defects following RNAi, including tissue regression following regeneration, lysis, caudal blastema abnormalities, photoreceptor defects, and paralysis (Table 7 of the incorporated PCT International Application PCT/US2004/037475 (corresponding to PCT International Publication WO 2005/047300 A2 of May 26, 2005)). These 143 genes were inhibited by RNAi in 20 animals each. Eight animals were left intact, fed five times over four weeks, and observed 3-4 times a week for 10 weeks to assess the role of these genes in tissue homeostasis (FIG. 3D). Twelve animals from the RNAi of each of the 143 genes were amputated following the protocol described for the screen (FIG. 3B). Of these, six were fixed 16 or 24 hours following amputation and labeled with αH3P (see above), and the rest observed as a control for the effectiveness of the RNAi treatment (FIG. 3D). The numbers of dividing cells were compared to those of control RNAi (C. elegans unc-22), amputated animals. RNAi of 111 of these 143 genes conferred robust defects that define the major planarian homeostasis phenotypes (Table 8 of the incorporated PCT International Application PCT/US2004/037475 (corresponding to PCT International Publication WO 2005/047300 A2 of May 26, 2005), FIGS. 6B-G). Surprisingly, there was great diversity in the patterns of lesion formation and of tissue regression in intact, RNAi animals, demonstrating the complex manner in which perturbation of different genes affects homeostasis in planarians (FIGS. 6B-G). For animals from the RNAi of each gene studied, FIGS. 7A-D compares the tissue homeostasis and neoblast proliferation results to the size of the blastema obtained following RNAi and amputation (see below).

Genes that regulate the control of cell turnover by neoblasts. Genes that confer RNAi phenotypes in intact adult animals similar to irradiated intact animals, and that are needed for regeneration, likely are needed for all aspects of neoblast functioning. Irradiated animals left intact displayed reproducible homeostasis defects: tissue regression within eight days (FIG. 6A), curling within 15 days (FIG. 6A), and lysis. The tissue anterior to the photoreceptors, where regression is typically observed, is normally incapable of regeneration²⁴ and is constantly replaced by neoblast progeny¹⁹. RNAi of many genes caused defects similar to those observed in irradiated, intact animals; and these genes may be needed for neoblast function (Table 8 of the incorporated PCT International Application PCT/US2004/037475 (corresponding to PCT International Publication WO 2005/047300 A2 of May 26, 2005), FIGS. 6B, C). Tissue regression and curling tend to appear together in RNAi experiments (32 out of 45 cases in which regression or curling was observed, P<0.0001) as well as with lysis (29/32), suggesting a common underlying defect (Table 8 of the incorporated PCT International Application PCT/US2004/037475 (corresponding to PCT International Publication WO 2005/047300 A2 of May 26, 2005), FIG. 7C). Decrease of αH3P-labeled cells following amputation correlates with curling and regression defects in intact animals (FIG. 7C). Genes that cause regression and curling following RNAi in intact animals tend to be needed for regeneration (26 of 32 genes, P<0.0005) indicating these genes may be required for all neoblast functions (FIG. 7C). Of the 66 genes in this study that were needed for regeneration (BLST(0/0.5)), RNAi of 31 of the genes caused intact animals to display tissue regression and RNAi of 28 of the genes caused intact animals to curl, indicating that only about half of the genes needed for regeneration may be needed for neoblast function in homeostasis. Among the 47 genes that caused curling and/or regression in intact animals following RNAi are 21 genes predicted to encode proteins involved in translation or metabolism, 2 genes in vesicle trafficking, 3 genes in cell cycle, 3 genes in chromatin factors, 1 gene in cytoskeletal protein, 4 genes in RNA-binding factors, 1 gene similar to a disease protein, 1 gene in protein transport, 2 genes in RNA splicing, 3 genes in signal transduction proteins, and 6 genes with unknown functions (Table 8 of the incorporated PCT International Application PCT/US2004/037475 (corresponding to PCT International Publication WO 2005/047300 A2 of May 26, 2005)). This gene set provides a profile of gene functions likely required for the functioning of neoblasts.

Genes needed specifically for rogation. Genes that are needed for regeneration also tend to be needed for homeostasis (P<0.005) (FIG. 7B). However, RNAi of 33 out of 143 genes conferred no or only minor defects in intact animals (Table 8 of the incorporated PCT International Application PCT/US2004/037475 (corresponding to PCT International Publication WO 2005/047300 A2 of May 26, 2005), FIG. 7B). Twenty-five of these 33 genes were associated with smaller than normal blastemas in two separate RNAi experiments (Table 8 of the incorporated PCT International Application PCT/US2004/037475 (corresponding to PCT International Publication WO 2005/047300 A2 of May 26, 2005)). One gene, HE.3.04D, is a candidate novel wound healing factor as it causes lysis following amputation when perturbed. Two genes, which are important for the formation of caudal blastemas (HE.4.06F, NBE.7.07H), are predicted to encode a novel protein and a nucleostemin-like GTPase (Tables 4, 8 of the incorporated PCT International Application PCT/US2004/037475 (corresponding to PCT International Publication WO 2005/047300 A2 of May 26, 2005)). At least four genes caused tissue regression following amputation and regeneration, and encode a transporter (NBE.2.08E), a potassium channel regulator (NBE.3.01 A), a myosin light chain (HE.2.11C), and an FKBP-like immunophilin (NBE.3.05F) (Id.) Genes needed for complete regeneration, but apparently not necessary for homeostasis, include those predicted to encode proteins similar to chondrosarcoma-associated protein 2 (NBE.3.11F), a DEAD box RNA-binding protein (HE.1.06D), SMAD4 (HE.3.03B), Baf53a (HE.3.10F), a topoisomerase (HE.3.05A), and a WW-domain protein (HE.3.02A) (Tables 4, 8). Some of these genes could identify signaling mechanisms that specifically activate neoblasts following wounding or control other processes needed for blastema generation and maintenance. One of these genes, SMAD4, stands apart as a gene necessary for any blastema formation, but dispensable for neoblast functioning in homeostasis. This observation indicates that TGF-β signalling may control regeneration initiation in planarians.

Genes needed for homeostasis but not basal neoblast functioning. RNAi of some genes caused robust, inviable homeostasis defects but did not block blastema formation following amputation (FIG. 7B). Additionally, not all genes required for homeostasis were needed for neoblast mitoses following amputation (FIG. 7B). Therefore, cellular events required for homeostasis need not be required for regeneration or always involve neoblast proliferation. A major category of homeostasis phenotypes involves the formation of a variety of types of lesions (FIGS. 6D-G). Genes that cause lesions in intact animals following RNAi do not have strong tendencies to block regeneration or neoblast proliferation following RNAi and amputation (FIG. 7D). This demonstrates that the cellular defects underlying lesion formation during homeostasis need not block regeneration or proliferation and that defects that block proliferation and regeneration need not cause lesions (FIG. 7D). Since irradiation of planarians does not result in lesions (FIG. 6A), lesions likely arise due to defects in differentiated cells. Of the 33 genes for which RNAi blocked regeneration, but did not cause regression or curling in intact animals, RNAi of 31 caused lesions (29/31 robustly) to develop in the intact animals. This striking correlation (P<0.0005) suggests that there may be two main categories of genes that are needed for regeneration and viability in adult animals: one category that regulates the functioning of the stem cells, and another that is necessary for the functioning of differentiated cells. These categories may not be mutually exclusive; e.g., some animals that had regression and/or curling also developed lesions (12/33). RNAi of 18 genes allowed regeneration of BLST≦2, but caused robust defects in intact animals. Fifteen of these are associated with lesion formation. Eight out of these 15 genes encode predicted signal transduction or transcription factors (as compared to 18 of 143 in the entire experiment) indicating these genes may function in the patterning and functioning of differentiated cells (Table 8 of the incorporated PCT International Application PCT/US2004/037475 (corresponding to PCT International Publication WO 2005/047300 A2 of May 26, 2005)).

### Example 14

### Blastema Morphology and Patterning Genes

A variety of blastema morphology and patterning-defective phenotypes were observed, including indented, pointed, and flat blastemas, as well as wide, faint, and no photoreceptors (Table 4 of the incorporated PCT International Application PCT/US2004/037475 (corresponding to PCT International Publication WO 2005/047300 A2 of May 26, 2005), FIGS. 4C, 4D). The molecular identities of genes thus inferred to be involved in these attributes of planarian blastema patterning can be found in Tables 4, 5 of the incorporated PCT International Application PCT/US2004/037475 (corresponding to PCT International Publication WO 2005/047300 A2 of May 26, 2005). Wild-type planarians are bilaterally symmetric with no known asymmetry²⁵. RNAi of five genes caused asymmetric regeneration of photoreceptors, indicating active mechanisms may exist for maintaining symmetry in animal species that lack asymmetry (Tables 4, 5 of the incorporated PCT International Application PCT/US2004/037475 (corresponding to PCT International Publication WO 2005/047300 A2 of May 26, 2005), FIG. 4D, FIG. 5F). Eighteen of the genes that caused regression (RGRS) following RNAi caused regression of blastemas, possibly the result of defects in blastema maintenance (Table 4, FIG. 4E). Surprising and novel phenotypes identify candidate properties of planarian biology for further exploration. For instance, ectopic neuronal material at the midline in H.68.4a Slit(RNAi) animals may serve to induce ectopic axis formation perpendicular to the original animal axis (Tables 4, 7, FIG. 5H). In another example, indented blastemas in HE.2.07D BMP1(RNAi) animals indicate BMP signaling may control regeneration of midline tissues (Table 4, FIGS. 4C, 5I). Surprising defects such as these not only illuminate the genetic control of specific aspects of planarian biology, but also indicate that undiscovered roles for known genes in understudied biological processes can be identified in planarians.

### Example 15

### Behavior Genes

Planarians locomote via the beating of ventral cilia, can move their body to turn and respond to objects by use of their muscular system, and control their behavior with bicephalic ganglia, two ventral nerve tracts, a variety of sensory systems, and a submuscular nervous plexus²⁵. RNAi of 44 genes conferred uncoordinated locomotion (36 robustly) with RNAi of two additional genes giving uncoordinated flipping (flp) (Tables 4, 5 of the incorporated PCT International Application PCT/US2004/037475 (corresponding to PCT International Publication WO 2005/047300 A2 of May 26, 2005)). Following the RNAi of some genes, such as a proprotein convertase-encoding gene (HE.2.02B), animals became completely paralyzed (Table 4). Five genes conferred blistering (BLI) and bloating (BLT) as well as lack of coordination following RNAi, including genes predicted to encode cytoskeletal proteins such as tubulins (HE.1.01H, HE.1.03G), a-spectrin (HE.1.08G), and rootletin (HE.1.02E) (Table 4, FIG. 4G). Since cilia-structures are needed for both locomotion and the excretory system, these genes may control cilia function^{25,26}. RNAi of four genes caused animals to become uncoordinated and to adopt an abnormal body posture, such as becoming flattened (flattened) following RNAi of a secretory granule neuroendocrine protein-encoding gene (HE.4.05F), or becoming narrower in the middle than at the ends (hourglass) following RNAi of a tropomyosin-encoding gene (NBE.1.12G) (Table 4 of the incorporated PCT International Application PCTIUS2004/037475 (corresponding to PCT International Publication WO 2005/047300 A2 of May 26, 2005), FIG. 4H). RNAi of one gene, predicted to encode a protein similar to a hepatocellular-associated antigen (NBE.8.11C), caused animals to stick to a surface and stretch their bodies out to a very thin morphology (stick&stretch) (Table 4 of the incorporated PCT International Application PCT/US2004/037475 (corresponding to PCT International Publication WO 2005/047300 A2 of May 26, 2005), FIG. 4H). RNAi of one gene, predicted to encode an outer dense fiber of sperm tails-like protein (NBE.8.03E), caused animals to move sideways to the right (sidewinder) (Table 2). Other genes associated with abnormal behavior are predicted to encode proteins including G-protein factors, transcription factors, and 12 novel proteins (Table 4 of the incorporated PCT International Application PCT/US2004/037475 (corresponding to PCT International Publication WO 2005/047300 A2 of May 26, 2005)). These results assign behavioral functions to an assortment of genes and identify functions for previously uncharacterized genes.

All references, including publications, patents, patent applications, and sequence accession numbers cited herein are hereby incorporated by reference to the same extent as if each reference were individually and specifically indicated to be incorporated by reference and were set forth in its entirety herein.

While this invention has been described in certain embodiments, the invention can be further modified within the spirit and scope of this disclosure. This application is therefore intended to cover any variations, uses, or adaptations of the invention using its general principles. Further, this application is intended to cover such departures from the disclosure as come within known or customary practice in the art to which this invention pertains and which fall within the limits of the appended claims.

### TABLES

TABLE 3
Summary of S. mediterranea RNAi screen results. Phenotype descriptors
and details can be found in Table 4. Since the phenotype of many
genes involved multiple defects, a particular gene may fall into
multiple phenotype categories. Each gene falls into only one functional
category. Homology details can be found in Table 4.

| Screen and | No. | | No. |
|---|---|---|---|
| phenotype details | Genes | Functional category | Genes |
| Total screened | 1065 | Translation | 39 |
| Total with phenotype | 240 | No match | 35 |
| No regeneration | 69 | Signal transduction | 29 |
| Limited regeneration | 35 | Metabolism | 19 |
| Reduced regeneration | 36 | Transcription/chromatin | 17 |
| Caudal blastema | 6 | RNA binding | 16 |
| Regression | 23 | Cytoskeleton | 13 |
| Curling | 48 | Protein degradation/ | 12 |
| Blastema morphology | 43 | protease | |
| Photoreceptors | 79 | Vesicle/protein | 11 |
| Lesions | 20 | trafficking | |
| Lysis | 76 | Novel with homology | 8 |
| Blisters &/or Bloating | 8 | Channels & transporters | 7 |
| Behavior | 44 | Cell cycle/DNA repair | 8 |
| Pigmentation | 8 | Neuronal | 7 |
| Antibody only | 20 | Disease | 6 |
| Other | 15 | RNA splicing/metabolism | 5 |
| | | Cell adhesion/ECM | 4 |
| | | Protein folding/stability | 4 |

### REFERENCES

1. Newmark, P. A. & Sanchez Alvarado, A. Not your father's planarian: a classic model enters the era of functional genomics. Nature Reviews Genetics 3, 210-219 (2002).

2. Reddien, P. W. & Sanchez Alvarado, A. Fundamentals of planarian regeneration. Ann. Rev. Cell Dev. Bio. (2004).

3. Nusslein-Volhard, C. & Wieschaus, E. Mutations affecting segment number and polarity in Drosophila. Nature 287, 795-801 (1980).

4. Brenner, S. The genetics of Caenorhabditis elegans. Genetics 77, 71-94 (1974).

5. Driever, W. et al. A genetic screen for mutations affecting embryogenesis in zebrafish. Development 123, 37-46 (1996).

6. Haffier, P. et al. The identification of genes with unique and essential functions in the development of the zebrafish, Danio rerio. Development 123, 1-36 (1996).

7. Fire, A. et al. Potent and specific genetic interference by double-standed RNA in Caenorhabditis elegans. Nature 391, 806-811 (1998).

8. Gonczy, P. et al. Functional genomic analysis of cell division in C. elegans using RNAi of genes on chromosome III. Nature 408, 331-6 (2000).

9. Fraser, A. G. et al. Functional genomic analysis of C. elegans chromosome I by systematic RNA interference. Nature 408, 325-30. (2000).

10. Kamath, R. S. et al. Systematic functional analysis of the Caenorhabditis elegans genome using RNAi. Nature 421, 231-7 (2003).

11. Sánchez Alvarado, A. & Newmark, P. A. Double-stranded RNA specifically disrupts gene expression during planarian regeneration. Proc. Natl. Acad. Sci. 96, 5049-5054 (1999).

12. Newmark, P. A., Reddien, P. W., Cebria, F. & Sánchez Alvarado, A. Ingestion of bacterially expressed double-stranded RNA inhibits gene expression in planarians. Proc. Natl. Acad. Sci. 100, 11861-5 (2003).

13. Timmons, L., Court, D. L. & Fire, A. Ingestion of bacterially expressed dsRNAs can produce specific and potent genetic interference in Caenorhabditis elegans. Gene 263, 103-12. (2001).

14. Casari, G. et al. Spastic paraplegia and OXPHOS impairment caused by mutations in paraplegin, a nuclear-encoded mitochondrial metalloprotease. Cell 93, 973-83 (1998).

15. Nishiguchi, K. M. et al. Defects in RGS9 or its anchor protein R9AP in patients with slow photoreceptor deactivation. Nature 427, 75-8 (2004).

16. Sakai, F., Agata, K., Orii, H. & Watanabe, K. Organization and regeneration ability of spontaneous supernumerary eyes in planarians-eye regeneration field and pathway selection by optic nerves. Zool. Sci. 17, 375-381 (2000).

17. Carpenter, K., Morita, M. & Best, J. Ultrastructure of the photoreceptor of the planarian Dugesia dorotocephala. I. Normal eye. Cell Tissue Res. 148, 143-158 (1974).

18. Inoue, T. et al. Morphological and functional recovery of the planarian photosensing system during head regeneration. Zool. Sci. 21, 275-83 (2004).

19. Newmark, P. & Sánchez Alvarado, A. Bromodeoxyuridine specifically labels the regenerative stem cells of planarians. Dev. Biol. 220, 142-53 (2000).

20. Hendzel, M. J. et al. Mitosis-specific phosphorylation of histone H3 initiates primarily within pericentromeric heterochromatin during G2 and spreads in an ordered fashion coincident with mitotic chromosome condensation. Chromosoma 106, 348-360 (1997).

21. Bardeen, C. R. & Baetjer, F. H. The inhibitive action of the Roentgen rays on regeneration in planarians. J. Exp. Zool. 1, 191-195 (1904).

22. Chandebois, R. Histogenesis and morphogenesis in planarian regeneration. Monogr. Dev. Biol. 11, 1-182 (1976).

23. Dubois, F. Contribution á 1 'ètude de la migration des cellules de règènèration chez les Planaires dulcicoles. Bull. Biol. Fr. Belg. 83, 213-283 (1949).

24. Morgan, T. H. Experimental studies of the regeneration of Planaria maculata. Arch. Entw. Mech. Org. 7, 364-397 (1898).

25. Hyman, L. H. The Invertebrates: Platyhelminthes and Rhynchocoela The acoelomate bilateia (McGraw-Hill Book Company Inc., New York, 1951).

26. Ishii, S. The ultrastructure of the protonephridial flame cell of the freshwater planarian Bdellocephala brunnea. Cell Tissue Res. 206, 441-9 (1980).

27. Adoutte, A. et al. The new animal phylogeny: reliability and implications. Proc Natl Acad Sci USA 97, 4453-6. (2000).

28. Oviedo, N. J., Newmark, P. A. & Sánchez Alvarado, A. Allometric scaling and proportion regulation in the freshwater planarian Schmidtea mediterranea. Dev. Dyn. 226, 326-33 (2003).

29. Sánchez Alvarado, A., Newmark, P. A., Robb, S. M. & Juste, R. The Schmidtea mediterranea database as a molecular resource for studying platyhelminthes, stem cells and regeneration. Development 129, 5659-65 (2002).

1. A method of attenuating expression of a target nucleotide sequence in a eukaryotic cell, said method comprising:
introducing double-stranded RNA (dsRNA) into the eukaryotic cell to attenuate expression of the target nucleotide sequence;
wherein the dsRNA comprises a nucleotide sequence that hybridizes under stringent conditions to the target nucleotide sequence; and
wherein said dsRNA is expressed from a vector containing one or more transcription terminators.

2. The method according to claim 1, wherein introducing the dsRNA into the eukaryotic cell comprises introducing an expression vector including at least one nucleotide sequence similar to the target nucleotide sequence; and wherein said vector produces the dsRNA in an amount sufficient to attenuate expression of the target nucleotide sequence when said at least one nucleotide sequence is transcribed.

3. The method according to claim 2, wherein transcription of the at least one nucleotide sequence is initiated in both sense and antisense directions; wherein the at least one nucleotide sequence is functionally linked to two transcriptional regulatory sequences; wherein said transcriptional regulatory sequences terminate transcription in both directions at points sufficient to form complementary transcripts, and wherein said complementary transcripts anneal to form said dsRNA.

4. The method according to claim 2, wherein said expression vector includes at least two nucleotide sequences; wherein said at least two nucleotide sequences produce upon transcription, respectively, at least two complementary RNA sequences; and wherein said RNA sequences anneal to form said dsRNA.

5. The method according to claim 2, wherein at least one of said at least two nucleotide sequences produces a hairpin upon transcription, and wherein said hairpin anneals to form said dsRNA.

6. The method according to claim 2, wherein said expression vector includes at least one transcription regulatory sequence that causes transcription to stop.

7. The method according to claim 6, wherein said expression vector includes at least two transcription regulatory sequences.

8. The method according to claim 1, wherein introducing the dsRNA into the eukaryotic cell comprises introducing an expression vector having at least two promoters into the eukaryotic cell; wherein said two promoters are oriented such that the nucleotide sequence that hybridizes to the target nucleotide sequence that is flanked between the promoters, and upon binding of an appropriate transcription factor to the two promoters, the two promoters are capable of initiating transcription of the nucleotide sequence that hybridizes to the target nucleotide sequence; and wherein transcription of said nucleotide sequence that hybridizes to the target nucleotide sequence is carried out under conditions effective to generate the dsRNA in an amount sufficient to attenuate expression of the target gene.

9. (canceled)

10. The method according to claim 2, wherein said expression vector includes a nucleotide sequence encoding at least one selectable marker.

11. The method according to claim 10, wherein said nucleotide sequence encoding the selectable marker encodes a kanamycin resistance gene.

12. The method according to claim 1, wherein the eukaryotic cell is selected from the group consisting of an undifferentiated stem cell, the progeny of an undifferentiated stem cell, an embryonic stem cell, an embryonic stem cell of a planarian origin, a plant, a vertebrate, an invertebrate, and other eukaryotic cells.

13. The method according to claim 1, wherein the eukaryotic cell is a planarian cell or a Caenorhabditis elegans or Schmidtea mediterranea.

14. The method according to claim 13, wherein the eukaryotic cell is planarian cell and further wherein introducing the dsRNA into the eukaryotic cell comprises:
cloning the nucleotide sequence that hydridizes to the target gene into an expression vector;
transforming a bacterial cell with the expression vector; and
placing the bacterial cell in contact with the planarian cell.

15. The method according to claim 14, wherein placing the bacterial cell in contact with the planarian cell comprises feeding the bacterial cell to a planarian organism.

16. The method according to claim 8, further comprising:
constructing a library of target nucleotide sequences cloned into an expression vector, thus producing a dsRNA library;
placing the dsRNA library into contact with a plurality of eukaryotic cells;
identifying members of the dsRNA library which confer a particular phenotype on an eukaryotic cell or otherwise cause a cellular change in the eukaryotic cell; and
determining the nucleotide sequence which corresponds to the library member that confers the particular phenotype or otherwise causes the cellular change in the eukaryotic cell.

17. A method of discovering a drug having an effect on a cell, said method comprising:
identifying a target gene which confers a phenotypically desirable response when inhibited by RNAi with the method according to claim 16;
identifying agents capable of inhibiting or activating expression of the target gene or inhibiting or activating the activity of an expression product of the target gene;
conducting therapeutic profiling of the identified agents, or further analogs thereof, for efficacy and toxicity in animals; and
formulating a pharmaceutical preparation including one or more identified agents as having an acceptable therapeutic profile.

18. The method according to claim 1, wherein introducing the dsRNA into the eukaryotic cell comprises introducing a hairpin nucleic acid in an amount sufficient to attenuate expression of the target gene into the eukaryotic cell.

19. A method of alleviating pest infestation or infection of an organism, said method comprising:
identifying a target gene of said pest that is critical for the pest's survival, growth, proliferation or reproduction with the method according to claim 16;
cloning a nucleotide sequence that hybridizes under stringent conditions to the target gene or a fragment thereof in a vector capable of expressing dsRNA; and
placing said vector into contact with the organism under conditions effective to alleviate the pest infestation or infection.

20. The method according to claim 19, wherein one or more tissue specific promoters are used to limit expression of said dsRNA to one or more specific organism tissues.

21. The method according to claim 19, wherein said pest is selected from the group consisting of a nematode worm, an insect, a bacterium, a fungi, and a planarian.

22. The method according to claim 19, wherein said target gene sequence of said pest is not a genomic sequence from said organism.

23. The method according to claim 19, wherein said organism is an animal or a plant.

24. The method according to claim 12, wherein said embryonic stem cell is the result of nuclear transfer.

25. The method according to claim 24,
wherein a donor nuclei is transferred to a previously modified recipient oocyte; and
wherein said recipient oocyte is modified by introducing one or more dsRNAs into said oocyte under conditions effective to modify said oocyte.

26. The method according to claim 25, wherein an embryonic stem cell obtained from said modified recipient oocyte, or the differentiated progeny thereof, is further modified by introducing one or more dsRNAs into the cell under conditions effective to modify said stem cell or said differentiated progeny thereof.

27. The method according to claim 26, wherein modification of said recipient oocyte comprises one or more changes in the expression of a gene or protein of the oocyte effective to prevent successful implantation of an embryo derived from the modified oocyte.

28. The method according to claim 26, wherein said alteration is carried out under conditions effective to decrease or eliminate Major Histocompatibility Complex (MHC) expression.

29. The method according to claim 26, wherein said alteration is carried out under conditions effective to decrease or eliminate the expression of one or more genes required for viral or bacterial infection of said cell.

30. The method according to claim 26, wherein said alteration is carried out under conditions effective to decrease or eliminate the expression of one or more genes required for viral or bacterial infection of said cell.

31. (canceled)

32. A dsRNA for inhibiting expression of a gene, said dsRNA comprising:
a first nucleotide sequence that hybridizes under stringent conditions to a target sequence, wherein the target sequence is complementary to said first nucleotide sequence, and wherein said stringent conditions include a wash step of 0.2×SSC at 65° C.

33. A hairpin nucleic acid for inhibiting expression of a target gene, said hairpin nucleic acid comprising the dsRNA of claim 32.

34. A cell comprising the dsRNA of claim 32.

35. The dsRNA according to claim 32, wherein said first nucleotide sequence comprises at least 20 nucleotides.

36. The dsRNA according to claim 32, wherein said first nucleotide sequence comprises a number of nucleotides, said number selected from the group consisting of at least 25 nucleotides, at least 100 nucleotides, and at least 400 nucleotides.

37. The dsRNA according to claim 32, wherein said first nucleotide sequence comprises a eukaryotic gene.

38. The dsRNA according to claim 37, wherein the eukaryotic gene is of animal origin.

39. The dsRNA according to claim 32, wherein said first nucleotide sequence is substantially identical to a nucleotide sequence which corresponds to at least one non-coding sequence of at least one eukaryotic gene, wherein the at least one eukaryotic gene is not fund in a genome of a host.

40. An expression vector comprising the dsRNA of claim 32.

41. The expression vector of claim 40, further comprising one or more promoters oriented relative to the a first nucleotide sequence such that the one or more promoters are capable of initiating transcription of said target gene DNA sequence to produce dsRNA.

42. The expression vector according to claim 40, wherein two promoters flank the first nucleotide sequence.

43. The expression vector according to claim 42, wherein the first nucleotide sequence is flanked by two transcription termination sequences.

44. The expression vector according to claim 32, comprising at least one selectable marker.

45. (canceled)

46. The expression vector according to claim 40, comprising transcription terminators.

47. The cell of claim 34, wherein the cell comprises a bacterial cell, a transgenic eukaryotic cell, or a germline cell.

48. The cell of claim 47, wherein said transgene is integrated into a chromosome of the transgenic eukaryitic cell.

49. The cell of claim 48, wherein the dsRNA construct is conditionally expressed.

50. The cell of claim 48, wherein the dsRNA construct is transiently transfected.

51. The expression vector of claim 40, wherein the expression vector comprises a plasmid identified as pDONR dT7.

52. A library of first nucleotide sequences comprising the vector of claim 51.

53. The library of claim 52, further comprising a plurality of first nucleotide sequences that hybridize under stringent conditions to a plurality of target sequences.
